(19) United States
(12) Patent Application Publication
(10) Pub. No.: US 2026/0315028 A1
(43) Pub. Date: Oct. 15, 2026

(54) ULTRASOUND APPARATUS AND METHOD

(71) Applicant: University of Dundee, Dundee (GB)

(72) Inventor: Luca Antonio Forte, Dundee (GB)

(73) Assignee: University of Dundee, Dundee (GB)

(21) Appl. No.: 19/484,858

(22) PCT Filed: May 17, 2024
(86) PCT No.: PCT/GB2024/051300

§ 371 Date: Nov. 14, 2025

(30) Foreign Application Priority Data
May 19, 2023 (GB) 2307520.3

(51) Int. Cl. A61B 8/08; A61B 8/00; A61B 8/13

(52) U.S. Cl. CPC A61B 8/0825; A61B 8/13; A61B 8/4281; A61B 8/4488; A61B 8/5207

(57) ABSTRACT

An ultrasound computed tomography apparatus comprises an imaging area configured to accommodate a medium to be imaged; an array of ultrasound elements arranged around the imaging area, the ultrasound elements comprising a transmitter, and a plurality of receivers; respective receiver circuitry for each receiver of the plurality of receivers; and a processing resource configured to perform computed transmission tomography. The transmitter is configured to transmit through the imaging area a sequence of single-tone continuous-wave signals, the sequence of single-tone continuous-wave signals comprising at least one single-tone continuous-wave signal having a first frequency and at least one single-tone continuous-wave signal having a second, different frequency. Each of the plurality of receivers is configured to receive the sequence of single-tone continuous-wave signals that has been transmitted through the imaging area. The respective receiver circuitry for each of the plurality of receivers is configured to mix each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal. The processing circuitry is configured to process the obtained in-phase and quadrature values by computed transmission tomography to obtain an image of the imaging area.

### FIELD

The present disclosure relates to an ultrasound apparatus and method for computed tomography ultrasound imaging, for example for ultrasound imaging of the breast.

### BACKGROUND

Breast cancer is the most common cancer amongst women. It accounts for one-third of cancers diagnosed and it represents the third leading cause of cancer death amongst women. A survival rate for breast cancer critically depends on an early diagnosis and dramatically falls for diagnoses at a later stage. Despite many countries having specific programs for breast screening, the rate of participation is typically low, and thus many cases are undetected until a later stage.

X-ray mammography may be considered to represent a gold standard for initial screening for breast cancer. Although the radiation dose absorbed after a conventional X-ray radiographic mammographic examination is typically very low, women who undergo X-ray radiographic mammographic examination may absorb unnecessary ionizing radiation. Also, a common issue with mammography is that in dense breast, cancer lesions may be missed. This means that, for women with dense breast, many cancer cases are undiagnosed. Thus, improving the detection rate of such cases is important as it may significantly lower the mortality rate.

It is known to perform breast imaging using ultrasound. In handheld ultrasound, a handheld transducer sends ultrasound signals to a region of the body and receives reflected ultrasound signals from the region of the body.

A typical handheld ultrasound system is a pulsed system with direct RF sampling. One linear array comprises, for example, 128 or 256 elements. All elements are configured both to transmit ultrasound signals and to receive ultrasound signals. Only reflection data, which may also be referred to as pulse-echo data, is collected. Reflected signals are collected at a single side, for example a single side of the breast, which is the same side from which ultrasound signals are transmitted. Image reconstruction may be performed using a conventional delay-and-sum beamforming algorithm, often in the time domain.

For screening purposes, the widespread adoption of handheld ultrasound is considered not to be feasible. Handheld ultrasound is operator dependent in that an operator is required to hold the handheld transducer and to select positions and settings for the ultrasound imaging. Handheld ultrasound has a limited footprint, which may hinder the imaging of the whole breast.

It is desirable to provide a safe and operator-independent method of ultrasound imaging of the breast for the diagnosis of breast cancer.

### SUMMARY

According to an aspect of the present disclosure, there is provided an ultrasound computed tomography method comprising: transmitting through a medium, by a transmitter positioned on a first side of the medium, a sequence of single-tone continuous-wave signals, the sequence of single-tone continuous-wave signals comprising at least one single-tone continuous-wave signal having a first frequency and at least one single-tone continuous-wave signal having a second, different frequency; receiving, by each of a plurality of receivers positioned at an opposite side of the medium, the sequence of single-tone continuous-wave signals that has been transmitted through the medium; for each of the plurality of receivers, mixing each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and processing the obtained in-phase and quadrature values by computed transmission tomography to obtain an image of the medium.

The medium may comprise breast tissue. The medium may comprise human or animal tissue. The medium may comprise at least part of a breast, a brain, a leg, an arm, a chest, a neck or a testicle.

The transmitter and receivers may form part of an array of ultrasound elements arranged around an imaging area in which the medium is positioned.

The array may be a circular array. The array may be a part-circular array.

The method may further comprise moving the transmitter and receivers relative to the medium and perpendicular to a plane of the transmitter and receivers. The moving may be by a succession of positional increments. The method may further comprise, for each of the positional increments, performing a respective further transmission of the sequence of single-tone continuous-wave signals and processing respective in-phase and quadrature values obtained from said further transmission by computed transmission tomography to obtain a respective two-dimensional image. The obtaining of the image of the medium may comprise combining said two-dimensional images to obtain a three-dimensional volume.

The array may be a three-dimensional array comprising a plurality of two-dimensional arrays. The two-dimensional arrays may be circular. The two-dimensional arrays may be part-circular. The two-dimensional arrays may be of different sizes. The two-dimensional arrays may be circular arrays having different radii.

The array may comprise first elements having first characteristics and second elements having second, different characteristics. The different characteristics may comprise at least one of a different height, a different width, a different thickness. The different characteristics may comprise different focusing properties. The different characteristics may comprise at least one of different foci, different beamwidths, different frequency bandwidths, different transmission frequencies. The first elements may be associated with a first type of acoustic lens and the second elements may be associated with a second, different type of acoustic lens having different acoustic properties.

The first elements may transmit in a first, lower frequency bandwidth and the second elements may transmit in a second, higher frequency bandwidth. The first, lower frequency bandwidth may be, for example, between 100 KHz and 500 kHz, between 200 KHz and 300 kHz or between 300 kHz and 500 kHz or between 200 kHz and 500 kHz, between 200 kHz and 300 kHz or between 300 kHz and 500 kHz. The second, higher-frequency bandwidth may be, for example, between 500 kHz and 1 MHz, between 1 MHz and 3 MHz, between 1 MHz and 2 MHz or between 2 MHz and 3 MHz.

Ultrasound elements of different two-dimensional arrays may have different characteristics. A first two-dimensional array may comprise first elements having first characteristics and a second two-dimensional array may comprise second elements having second, different characteristics.

The array may be arranged on a convex surface, for example a bowl-shaped surface.

The array may comprise at least one pair of opposing arrays. The opposing arrays may be two-dimensional arrays. The method may further comprise rotating the at least one pair of opposing arrays around the medium. The opposing arrays may be positioned at different sides of the medium, for example at opposite sides of the medium. The opposing arrays may face each other. The opposing arrays may be positioned such that signals transmitted from a first array of the opposing arrays is transmitted through the medium and received by a second array of the opposing arrays. For each pair of opposing arrays, a distance between the opposing arrays may be controlled mechanically and/or electronically. The distance between the opposing arrays may be varied, for example varied in dependence on elevation.

The at least one pair of opposing arrays may comprise a first pair of opposing arrays comprising ultrasound elements having first characteristics and a second pair of opposing arrays comprising ultrasound elements having second, different characteristics. The first elements may transmit in a first, lower frequency bandwidth and the second elements may transmit in a second, higher frequency bandwidth.

The first frequency may be between 100 KHz and 20 MHz. The second frequency may be between 100 KHz and 20 MHz. The first frequency may be between 300 kHz and 3 MHz. The second frequency may be between 300 kHz and 3 MHz. Each of the first frequency and second frequency may be greater than 100 kHz, optionally 200 kHz, further optionally 300 kHz, further optionally 500 kHz, further optionally 1 MHz. Each of the first frequency and second frequency may be less than 20 MHz, optionally 10 MHz, further optionally 5 MHz, further optionally 3 MHz.

The sequence of single-tone continuous-wave signals may comprise single-tone continuous-wave signals having a pre-programmed set of frequencies.

Each frequency of the pre-programmed set of frequencies may be greater than 100 kHz, optionally 200 kHz, further optionally 300 kHz, further optionally 500 kHz, further optionally 1 MHz. Each frequency of the pre-programmed set of frequencies may be less than 20 MHz, optionally 10 MHz, further optionally 5 MHz, further optionally 3 MHz.

The sequence of single-tone continuous-wave signals may comprise two or more single-tone continuous-wave signals having the first frequency. The method may further comprise combining the in-phase values for said two or more single-tone continuous-wave signals to obtain a combined in-phase value for the first frequency and combining the quadrature values for said two or more single-tone continuous-wave signals to obtain a combined quadrature value for the first frequency.

The processing of the obtained in-phase and quadrature values by computed transmission tomography may comprise a model-based image reconstruction. The processing of the obtained in-phase and quadrature values by computed transmission tomography may use an iterative algorithm based on numerical optimization of a cost function. The iterative algorithm may comprise determining a difference between measured or obtained data and synthetic data. The iterative algorithm may comprise iteratively updating an estimate for a physical quantity until convergence is reached. The processing of the obtained in-phase and quadrature values by computed transmission tomography may comprise processing the obtained in-phase and quadrature values by computed transmission tomography with model-based, iterative reconstruction techniques with or without phase-encoding.

According to an aspect of the present disclosure, which may be provided independently, there is provided an ultrasound computed tomography apparatus comprising: an imaging area configured to accommodate a medium to be imaged; an array of ultrasound elements arranged around the imaging area, the ultrasound elements comprising a transmitter positioned on a first side of the imaging area, and a plurality of receivers positioned on an opposite side of the imaging area; respective receiver circuitry for each receiver of the plurality of receivers; and a processing resource configured to perform computed transmission tomography; wherein the transmitter is configured to transmit through the imaging area a sequence of single-tone continuous-wave signals, the sequence of single-tone continuous-wave signals comprising at least one single-tone continuous-wave signal having a first frequency and at least one single-tone continuous-wave signal having a second, different frequency; each of the plurality of receivers is configured to receive the sequence of single-tone continuous-wave signals that has been transmitted through the imaging area; the respective receiver circuitry for each of the plurality of receivers is configured to mix each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and the processing circuitry is configured to process the obtained in-phase and quadrature values by computed transmission tomography to obtain an image of the imaging area.

The array of ultrasound elements may comprise a plurality of transceivers each configured to transmit and receive ultrasound signals. The transmitter may be a first transceiver of the plurality of transceivers. The receivers may be further transceivers of the plurality of transceivers.

The array may be a circular array. The array may be a part-circular array.

The array may be moveable relative to the medium and perpendicular to a plane of the transmitter and receivers. The array may be moveable by a succession of positional increments relative to the medium and perpendicular to the plane of the transmitter and receivers.

The array may be a three-dimensional array comprising a plurality of two-dimensional arrays. The two-dimensional arrays may be circular. The two-dimensional arrays may be part-circular. The two-dimensional arrays may be of different sizes. The two-dimensional arrays may be circular arrays having different radii.

Ultrasound elements of different two-dimensional arrays may have different characteristics.

The array may comprise a plurality of two- or three-dimensional sub-arrays, wherein each two- or three-dimensional sub-array is configured to transmit and receive ultrasonic signals in different frequency bandwidths.

The array may be wearable and/or may be provided as a wearable patch. For example, the array may comprise a flexible substrate or membrane, wherein the transmitter and the plurality of receivers are mounted on, attached to, or embedded in, the flexible substrate or membrane. The array may comprise an adhesive layer for attaching the flexible substrate or membrane to the medium.

Each two- or three-dimensional sub-array may comprise a corresponding flexible substrate or membrane, wherein the transmitter and the plurality of receivers of the two- or three-dimensional sub-array are mounted on, attached to, or embedded in, the corresponding flexible substrate or membrane. Each two- or three-dimensional sub-array may comprise an adhesive layer for attaching the corresponding flexible substrate or membrane to the medium.

The array may be arranged on a convex surface, for example a bowl-shaped surface.

The array may comprise at least one pair of opposing two-dimensional arrays. The method may further comprise rotating the at least one pair of opposing two-dimensional arrays around the medium.

The at least one pair of opposing two-dimensional arrays may comprise a first pair of opposing two-dimensional arrays comprising ultrasound elements having first characteristics and a second pair of opposing two-dimensional arrays comprising ultrasound elements having second, different characteristics.

The receiver circuitry may comprise a mixer stage configured to mix each received single-tone continuous-wave signal of the sequence of single-tone continuous-wave signals with the respective single-frequency mixer signal having the same frequency as said single-tone continuous-wave signal to obtain the respective in-phase value and a respective quadrature value for said single frequency continuous-wave signal.

The receiver circuitry may further comprise a low-noise amplifier configured to amplify received signals. The receiver circuitry may further comprise a pair of low-pass filters to remove frequencies above a threshold frequency. The receiver circuitry may further comprise a pair of analog to digital converters to digitize the in-phase and quadrature values.

Each analog to digital converter may have a bit depth of at least 16 bits. Each analog to digital converter may have a bit depth of at least 20 bits. Each analog to digital converter may have a bit depth of at least 24 bits. Each analog to digital converter may be a bit depth of less than 64 bits, optionally less than 32 bits, further optionally less than 28 bits. Each analog to digital converter may have a bit depth of 24 bits.

A sampling rate of each analog to digital converter may be between 1 kHz and 1024 kHz or may be between 1 kHz and 1000 KHz. A sampling rate of each analog to digital converter may be greater than 1 kHz, optionally greater than 200 kHz, further optionally greater than 500 kHz. A sampling rate of each analog to digital converter may be less than 1000 kHz, optionally less than 750 kHz, further optionally less than 500 kHz. A sampling rate of each analog to digital converter may be 128 kHz. A sampling rate of each analog to digital converter may be 256 kHz. A sampling rate of each analog to digital converter may be 512 kHz.

The ultrasound computed tomography apparatus may further comprise a controller. The controller may be configured to determine and/or store at least one of a respective voltage, a respective phase and a respective transmission time for each of the single-tone continuous-wave signals. The controller may be configured to determine and/or store a respective integration time for each of the single-tone continuous-wave signals.

At least one of the respective voltages, respective phases and respective transmission times may be programmable. At least one of the respective voltages, respective phases and respective transmission times may be frequency-dependent. The respective integration times may be programmable. The respective integration times may be frequency-dependent.

The ultrasound computed tomography apparatus may further comprise a signal generator configured to generate the sequence of single-tone continuous-wave signals.

According to an aspect of the present disclosure, which may be provided independently, there is provided an ultrasound computed tomography method comprising:
transmitting through a medium, by a transmitter, a sequence of single-tone continuous-wave signals, the sequence of single-tone continuous-wave signals comprising at least one single-tone continuous-wave signal having a first frequency and at least one single-tone continuous-wave signal having a second, different frequency;
receiving, by each of a plurality of receivers, the sequence of single-tone continuous-wave signals that has been transmitted through the medium;
for each of the plurality of receivers, mixing each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and
processing the obtained in-phase and quadrature values by computed transmission tomography to obtain an image of the medium.

The transmitter may be positioned to a first side of the medium and the plurality of receivers may be positioned to a second side of the medium, wherein the second side of the medium is different to the first side of the medium.

The second side of the medium may be opposite, and optionally also lateral, to the first side of the medium.

The transmitter may be positioned on a first surface in space and the plurality of receivers may be positioned on a second surface in space. At least one of the first and second surfaces may be planar. At least one of the first and second surfaces may be curved. At least one of the first and second surfaces may be concave. The first and second surfaces may be curved towards one another. At least one of the first and second surfaces may have circular symmetry, cylindrical symmetry, or axial symmetry, relative to a direction of transmission of the sequence of single-tone continuous-wave signals. The first surface may define at least part of a first hemisphere. The second surface may define at least part of a second hemisphere.

The transmitter may be positioned on a first line in space and the plurality of receivers may be positioned on a second line in space. At least one of the first and second lines may be straight. At least one of the first and second lines may be curved. At least one of the first and second lines may be concave. The first and second lines may be curved towards one another. At least one of the first and second lines may be symmetric relative to a direction of transmission of the sequence of single-tone continuous-wave signals. The first line may define a first arc or at least part of a first semicircle. The second line may define a second arc or at least part of a second semicircle.

The processing of the obtained in-phase and quadrature values by computed transmission tomography may comprise using an iterative algorithm based on numerical optimization of a cost function. The processing of the obtained in-phase and quadrature values by computed transmission tomography may comprise processing the obtained in-phase and quadrature values by computed transmission tomography with model-based, iterative reconstruction techniques with or without phase-encoding.

The method may further comprise processing the obtained in-phase and quadrature values to obtain a B-mode image. The processing of the obtained in-phase and quadrature values to obtain a B-mode image may comprise delay and sum beamforming.

According to an aspect of the present disclosure, which may be provided independently, there is provided an ultrasound computed tomography apparatus comprising: an imaging area configured to accommodate a medium to be imaged; an array of ultrasound elements arranged around the imaging area, the ultrasound elements comprising a transmitter, and a plurality of receivers; respective receiver circuitry for each receiver of the plurality of receivers; and a processing resource configured to perform computed transmission tomography; wherein the transmitter is configured to transmit through the imaging area a sequence of single-tone continuous-wave signals, the sequence of single-tone continuous-wave signals comprising at least one single-tone continuous-wave signal having a first frequency and at least one single-tone continuous-wave signal having a second, different frequency; each of the plurality of receivers is configured to receive the sequence of single-tone continuous-wave signals that has been transmitted through the imaging area; the respective receiver circuitry for each of the plurality of receivers is configured to mix each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and the processing circuitry is configured to process the obtained in-phase and quadrature values by computed transmission tomography to obtain an image of the imaging area.

The transmitter may be positioned to a first side of the imaging area and the plurality of receivers may be positioned to a second side of the imaging area, wherein the second side of the imaging area is different to the first side of the imaging area.

The second side of the imaging area may be opposite, and optionally also lateral, to the first side of the imaging area.

The transmitter may be positioned on a first surface in space and the plurality of receivers may be positioned on a second surface in space. At least one of the first and second surfaces may be planar. At least one of the first and second surfaces may be curved. At least one of the first and second surfaces may be concave. The first and second surfaces may be curved towards one another. At least one of the first and second surfaces may have circular symmetry, cylindrical symmetry, or axial symmetry, relative to a direction of transmission of the sequence of single-tone continuous-wave signals. The first surface may define at least part of a first hemisphere. The second surface may define at least part of a second hemisphere.

The transmitter may be positioned on a first line in space and the plurality of receivers may be positioned on a second line in space. At least one of the first and second lines may be straight. At least one of the first and second lines may be curved. At least one of the first and second lines may be concave. The first and second lines may be curved towards one another. At least one of the first and second lines may be symmetric relative to a direction of transmission of the sequence of single-tone continuous-wave signals. The first line may define a first arc or at least part of a first semicircle. The second line may define a second arc or at least part of a second semicircle.

Features in one aspect may be applied as features in any other aspect, in any appropriate combination. For example, system features may be provided as method features or vice versa.

### BRIEF DESCRIPTION OF THE DRAWINGS

Embodiments of the invention are now described, by way of non-limiting example, and are illustrated in the following figures, in which:

FIG. 1A is a schematic illustration of a stepped-frequency continuous-wave ultrasound computed tomography (SFCW-USCT) system;

FIG. 1B is a schematic illustration of a ring of ultrasound transceiver elements of the SFCW-USCT system of FIG. 1A around a breast;

FIG. 2 is a schematic illustration of a transmitter and receiver path of the SFCW-USCT system of FIG. 1A;

FIG. 3 is a plot of voltage as a function of time used to create the single-frequency continuous-wave signal for use in the transmitter and receiver path shown in FIG. 2;

FIG. 4 shows plots of demodulated signals I and Q before and after low-pass filtering;

FIG. 5 shows a plot of a single tone transmitted signal and associated mixer waveforms;

FIG. 6 shows a plot of a received single tone after amplification by a low-noise amplifier;

FIG. 7 shows plots of demodulated signals I and Q in the time and frequency domains;

FIG. 8 shows the temporal evolution of low-pass filtered signals I_{filt} and Q_{filt};

FIG. 9 illustrates a method of integrating (after digitization) the low-pass filtered signals I_{filt} and Q_{filt} of FIG. 8;

FIG. 10 illustrates an alternative method of integrating (after digitization) the low-pass filtered signals I_{filt} and Q_{filt} of FIG. 8;

FIG. 11 illustrates the relationship between SNR and integration time;

FIG. 12 shows the absolute value of a complex sample d(Rx, Tx; fₖ) measured by a lateral receiver and by an opposite receiver during transmission of a single tone and the impact of a longer integration time for such an opposite receiver;

FIG. 13 illustrates a numerical phantom sample which includes multiple inclusions and the associated measurement geometry, wherein the phantom sample defines a spatial variation in the speed of sound in m/s and each inclusion imposes a spatially localised variation in the speed of sound in m/s;

FIG. 14 illustrates a numerical phantom sample defining a spatial variation in the speed of sound in m/s, the evolution of the reconstructed speed of sound image of the numerical phantom sample over five different frequency bandwidths, and the evolution of an associated cost function during an iterative image reconstruction method using the frequency domain data measured using SFCW-USCT system of FIG. 1A;

FIG. 15 illustrates the measurement of breast geometry to inform imaging protocols;

FIG. 16 illustrates a first alternative geometry of ultrasound transceiver elements in which ultrasound transceiver elements are arranged in a bowl;

FIG. 17 illustrates a second alternative geometry of ultrasound transceiver elements in which ultrasound transceiver elements are arranged in three circular rings;

FIG. 18 illustrates a third alternative geometry of ultrasound transceiver elements having arrays positioned at opposite sides of a medium;

FIG. 19 illustrates a fourth alternative geometry of ultrasound transceiver elements in which ultrasound transceiver elements are arranged in two circular rings, wherein each ring is used for imaging in different frequency bandwidths;

FIG. 20 is a schematic of a fifth alternative arrangement of ultrasound transceiver elements comprising wearable patches, wherein each patch includes an ultrasound transceiver array for 2D imaging;

FIG. 21 is a schematic of a sixth alternative arrangement of ultrasound transceiver elements comprising wearable patches, wherein each patch includes an ultrasound transceiver array for 3D imaging;

FIG. 22 is a schematic illustration of a hypothetical pulsed system with direct RF sampling, including a plot of voltage;

FIG. 23 shows plots of Fourier synthesis associated with the hypothetical pulsed system with direct RF sampling of FIG. 22;

FIG. 24 shows plots of Fourier analysis associated with the hypothetical pulsed system with direct RF sampling of FIG. 22;

FIG. 25 shows that the operation of the hypothetical pulsed system with direct RF sampling of FIG. 22 is mathematically equivalent to the operation of the SFCW-USCT system of FIG. 2; and

FIG. 26 compares the signal to noise ratio of signals measured using the hypothetical pulsed system with direct RF sampling of FIG. 22 with the signal to noise ratio of signals measured using the SFCW-USCT system of FIG. 2.

### DETAILED DESCRIPTION OF THE DRAWINGS

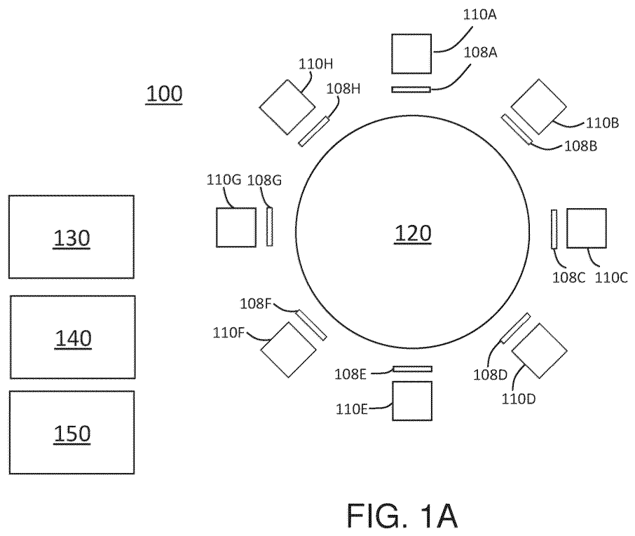

FIG. 1A is a simplified illustration of an ultrasound computed tomography system 100 in accordance with an embodiment. The ultrasound computed tomography system 100 is configured to perform computed transmission tomography in which ultrasound signals transmitted through a medium are processed to obtain an image.

The ultrasound computed tomography system 100 is a two-dimensional system. Elements are arranged in a two-dimensional array and data are collected at each of a plurality of fixed elevation locations, where elevation is a z direction orthogonal to an x-y imaging plane of the two-dimensional array. A whole breast is reconstructed as a 3D volume by stacking 2D images on top of each other.

The ultrasound computed tomography system 100 comprises an array of transceiver elements 110A, 110B . . . 110H arranged in a two-dimensional ring around an imaging area 120. Each of the transceiver elements 110A, 110B . . . 110H is a transducer element that is configured both to transmit and to receive ultrasound signals. In the example of FIG. 1, the system has a radius of 110 mm. In other embodiments, any suitable radius may be used.

The imaging area 120 is configured to accommodate a medium to be imaged, which in the present embodiment is breast tissue. In other embodiments, any suitable medium may be imaged, for example any appropriate type of human or animal tissue.

The array of transceiver elements 110A, 110B . . . 110H is moveable in a direction perpendicular to a plane of the two-dimensional ring formed by the transceiver elements. The array of transceiver elements 110A, 110B . . . 110H is therefore moveable with respect to a medium positioned in the imaging area 120.

FIG. 1A is a simplified drawing in which only 8 transceiver elements 110A, 110B . . . 110H are illustrated. In practice, the ultrasound computed tomography system comprises hundreds or thousands of transceiver elements, for example a single ring array may comprise 512 or 2048 transceiver elements.

A respective acoustic lens 108A, 108B . . . 108H is positioned in front of each of the transceiver elements 110A, 110B . . . 110H to focus waves into an imaging plane, and to receive waves only from the imaging plane.

Each transceiver element 110A, 110B . . . 110H is configured both to transmit ultrasound signals and to receive ultrasound signals. Each transceiver element therefore acts as both a transmitter element and a receiver element.

In other embodiments, a set of transmitter elements is used to transmit ultrasound signals and a set of receiver elements is used to receive ultrasound signals, where the receiver elements are different from the transmitter elements. In some such embodiments, the receiver elements may have different characteristics from the transmitter elements, for example a different size.

The transceiver elements 110A, 110B . . . 110H may also be referred to as ultrasound elements, array elements or transducer elements. Each transceiver element is configured to transmit a plurality of programmable frequencies, each of which is transmitted with a very narrow bandwidth, for example a bandwidth that is close to zero. Each transmitted frequency may be referred to as a respective single tone or single-tone continuous-wave signal or single-frequency continuous-wave signal.

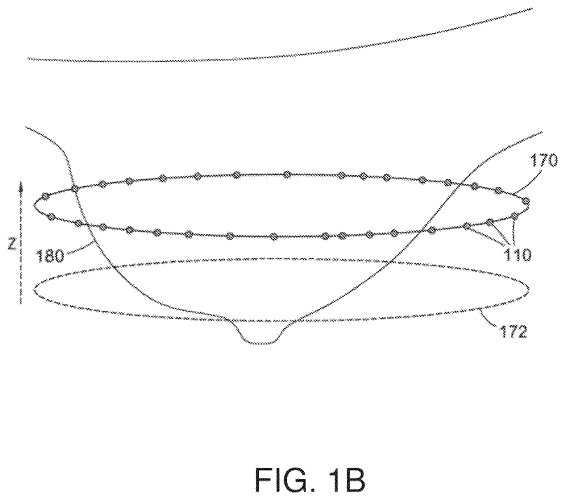

Each transceiver element 110A, 110B . . . 110H comprises a respective transducer 111A, 111B . . . 111H and respective receiver circuitry 112A, 112B . . . 112H (not shown in FIG. 1A). The receiver circuitry 112A, 112B . . . 112H comprises a low noise amplifier 113A, 113B . . . 113H; a mixer stage 114A, 114B . . . 114H and 115A, 115B . . . 115H; low-pass filters 116A, 116B . . . 116H and 117A, 117B . . . 117H; and analog to digital converters 118A, 118B . . . 118H and 119A, 119B . . . 119H (not shown in FIG. 1A). Components of the receiver circuitry 112E of transceiver element 110E are illustrated in FIG. 2. Other transceiver elements 110A, 110B . . . 110H comprise corresponding receiver circuitry 112A, 112B . . . 112H having the same components as those illustrated for transceiver circuitry 110E.

The ultrasound computed tomography system 100 further comprises a signal generator 130. The signal generator 130 is configured to generate a sequence of single-tone continuous-wave signals. Each of the single-tone continuous-wave signals comprises a respective sinusoidal continuous-wave signal having a respective predetermined frequency, as described further below. The signal generator 130 steps through a plurality of different predetermined frequencies in turn, generating a respective single-tone continuous-wave signal using a respective predetermined voltage, phase and transmission time for each of the predetermined frequencies.

The ultrasound computed tomography system 100 further comprises a controller 140 which is configured to control the signal generator 130 and to control which of the transceiver elements 110A, 110B . . . 110H is transmitting or receiving at a given time. The controller 140 may comprise one or more processors. In the present embodiment, the controller 140 stores the set of predetermined frequencies, voltages, phases and transmission times to be used in the generation of the sequence of single-tone continuous-wave signals. In other embodiments, the predetermined frequencies, voltages, phases and/or transmission times may be stored in any suitable data store.

In some circumstances, the frequencies, voltages, phases and/or transmission times may be selected by a user.

In the present embodiment, the system 100 is configured to operate in a clinical mode and in a research mode. In the clinical mode, the frequencies, voltages, phases and transmission times are stored and may not be changed by a clinical user, for example a radiographer. For example, a clinical user may not be permitted to change voltage levels for safety. For example, different imaging protocols may require different voltage levels, wherein the voltage level for any given imaging protocol is not editable by a clinical user.

In the research mode, a research user is allowed to fully program the system, which may include selecting frequencies and/or voltage amplitudes and/or phases and/or transmission times. Additionally, the research user may be allowed to program other parameters of the system 100, for example analog gains of the low noise amplifiers 113A, 113B . . . 113H. Transmission/dwell times of the single tones may be selected. In other embodiments, any suitable modes may be used, for example a clinical mode and/or research mode and/or screening mode and/or follow up mode and/or high frequency mode. Full programmability of system parameters may enable the use of multiple modes or imaging protocols.

In some embodiments, different modes use different frequencies. For example, a higher frequency mode may be used to provide higher resolution. In some embodiments, different modes use a different number of frequency steps, for example research mode may collect more frequency steps than clinical mode.

The ultrasound computed tomography system 100 further comprises a processing resource 150 configured to perform computed transmission tomography processing. The processing resource may comprise one or more processors.

The controller 140 and processing resource 150 may form part of one or more computing apparatuses, for example personal computers, servers or workstations. The one or more computing apparatuses may comprise one or more central processing Units (CPU) and/or Graphical Processing Units (GPU). For example, the controller 140 may be implemented in a CPU by means of a computer program having computer-readable instructions that are executable to perform the method of the embodiment and/or at least part of the controller 140 may be implemented as one or more ASICs (application specific integrated circuits) or FPGAs (field programmable gate arrays). The processing resource 150 may be implemented by a CPU and/or GPU by means of a computer program having computer-readable instructions that are executable to perform the method of the embodiment. In some embodiments, at least part of the receiver circuitry 112A, 112B . . . 112H and/or other electronics of the system 100 may be implemented as one or more ASICs (application specific integrated circuits) or FPGAs (field programmable gate arrays).

In use, a medium is positioned within the imaging area 120. In the present embodiment, the medium is the breast of a subject to be imaged. The two-dimensional ring-shaped array of transceiver elements 110A, 110B . . . 110H is positioned around the breast in a first position. In the present embodiment, the first position is a position closest to the subject's breastbone and the ring-shaped array scans in a direction away from the subject's chest. In other embodiments, the first position is the position furthest from the chest, for example a position that is aligned with a nipple, and the ring-shaped array scans in a direction towards the chest.

Each transceiver element 110A, 110B . . . 110H of the two-dimensional ring array is driven in turn described below to transmit a sequence of single-tone continuous-wave signals. After propagating in a medium, each single-tone continuous-wave signal is recorded by another transceiver element 110A, 110B . . . 110H. The signal is recorded by a plurality of elements in parallel.

A transmission by a first transceiver element 110A is described below. It is noted that a corresponding transmission is also made by each of the other transceiver elements 110B, 110C . . . 110H. Once all transceiver elements 110A, 110B . . . 110H have transmitted, the array is repositioned and the transmitting by each of the transceiver elements 110A, 110B . . . 110H is repeated. The repositioning and transmissions are repeated until the entire breast has been imaged.

FIG. 1B illustrates a ring array 170 comprising a plurality of transceiver elements 110 positioned around a breast 180. A dotted line 172 indicates a different position for the ring array. A direction of movement of the ring array is denoted as z in FIG. 1B.

FIG. 2 shows a transmitter and a receiver path for a stepped-frequency architecture in accordance with an embodiment. FIG. 2 illustrates an example of transmitting using a first transceiver element Tx and receiving using a second, different transceiver element Rx which is positioned opposite to the first transceiver element Tx in the array.

The controller 140 instructs the signal generator 130 to generate a sequence of single-tone continuous-wave signals. Each single-tone continuous-wave signal is a continuous transmission of a pure tone at a respective fixed frequency fₖ within a bandwidth of the transducer 111A. Each single-tone continuous-wave signal has a respective predetermined voltage, phase and transmission times. In the present embodiment, the controller 140 stores a set of predetermined frequencies fₖ and their associated voltages, phases and transmission times. The signal generator 130 steps through the set of different predetermined frequencies in turn. For each of the predetermined frequencies, the signal generator generates a respective single-tone continuous-wave signal that is generated using the associated voltage, phase and transmission time. The predetermined frequencies may be pre-programmed into the system. In some circumstances, the predetermined frequencies may be selected by a user.

In the case of transmission by the first transceiver element 110A, the controller 140 instructs the first transceiver element 110A to operate in a transmitting mode. The controller 140 instructs the signal generator 130 to provide the sequence of single-tone continuous-wave signals to the first transceiver element 112A, by first providing a first single-tone continuous-wave signal having a first frequency for a first transmission time, then providing a second single-tone continuous-wave signal having a second frequency for a second transmission time after transmission of the first single-tone continuous-wave signal is over, then subsequently providing any further single-tone continuous-wave signals one at a time, each having a respective frequency and provided for a respective transmission time. The transmission times may also be referred to as dwell times. Each single-tone continuous-wave signal may have a different transmission time. The controller 140 instructs the remaining, non-transmitting transceiver elements 110B, 110C . . . 110H to operate in a receiving mode. For each of the single-tone continuous-wave signals, the remaining transceiver elements 110B, 110C . . . 110H receive the single-tone continuous-wave signal. Each single-tone continuous-wave signal has a respective fixed frequency.

The signal generator 130 provides the each of the sequence of single-tone continuous-wave signals in turn to the first transceiver element 110A for transmission. For each of the single-tone continuous-wave signals, the transducer 111A of the first transceiver 110A converts the single-tone continuous wave signal from an electrical signal to an ultrasound signal which is transmitted into the medium 160. The ultrasound signal may also be referred to as an acoustic wave.

The following discussion describes in detail the transmitting, receiving and processing of one of the single-tone continuous-wave signals of the sequence of single-tone continuous-wave signals. The single-tone continuous-wave signal has a fixed frequency fₖ. Similar transmitting, receiving and processing occurs for each of the single-tone continuous-wave signals transmitted by the transmitter.

The single-tone continuous-wave signal may be written as a continuous transmission having a sinusoidal waveform sin(2πfₖt). The transducer 111A converts the single-tone continuous-wave signal from an electrical signal to an ultrasound signal.

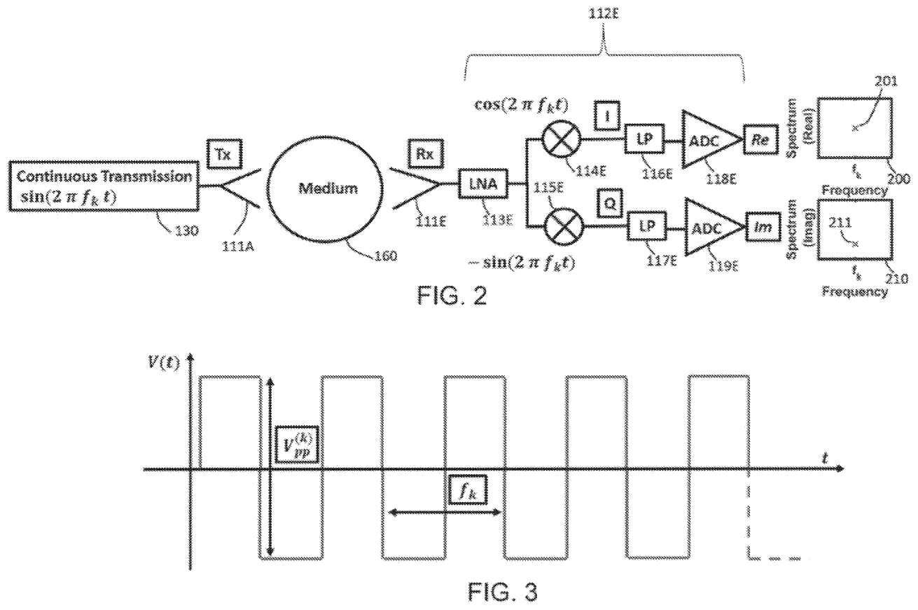

FIG. 3 is a plot of voltage versus time used to create the single-tone continuous-wave signal having frequency fₖ. A peak-to-peak voltage is denoted as Vₚₚ^{(k)}. Since the duty cycle is 1, RF peak power and average power are equal. In the embodiment shown in FIG. 3, the voltage waveform is a square wave. In some embodiments, the voltage waveform has the shape of a mathematical sinusoidal function rather than a square wave.

The ultrasound signal is transmitted from the first transceiver element 110A and passes through an acoustic lens 108A (not shown in FIG. 2) into the medium 160 and at least part of the ultrasound signal is transmitted through the medium 160 for detection by one or more of the remaining, non-transmitting transceiver elements 110B . . . 110H. As will be described in more detail below, the ultrasonic signals received by one or more of the remaining, non-transmitting transceiver elements 110B . . . 110H may be used for reconstructing an image of the medium. Part of the ultrasound signal may be diffracted. Part of the ultrasound signal may be reflected.

The first transceiver element 110A may be positioned to a first side of the medium 160. The one or more of the remaining, non-transmitting transceiver elements 110B . . . 110H, which are used to detect the ultrasound signal, may be positioned to a second side of the medium, wherein the second side of the medium is different to the first side of the medium. The second side of the medium may be positioned generally opposite to the first side of the medium. For example, the second side of the medium may be positioned opposite to, and optionally also lateral to, the first side of the medium. The one or more of the remaining, non-transmitting transceiver elements 110B . . . 110H, which are used to detect the ultrasound signal, may be positioned opposite to the transmitting first transceiver element 110A, and optionally also lateral to, the transmitting first transceiver element 110A.

Each of the receiving transceiver elements 110B . . . 110H receives a respective received ultrasound signal as a result of the transmission by transceiver element 110A. An example of reception by components of transceiver element 110E, which is directly opposite the transmitting transceiver element 110A, is illustrated in FIG. 2.

In the simplified embodiment illustrated in FIG. 1A, all receiving elements 110B, 110C . . . 110H receive in parallel when a fixed element is transmitting. In practical embodiments, the system may include hundreds or thousands of receiving elements. In some such embodiments, a sub-set of the receiving elements receives in parallel, followed by a further, different sub-set of the receiving elements. In some embodiments, receiving circuitry is shared between elements. For example, a single receiving circuitry may be used to receive signals from two or more transducers. A number of channels used to receive may be lower than a number of elements, and multiplexing may be used.

Turning again to the example of FIG. 2, a transducer 111E receives the ultrasound signal that has passed through the medium 160 via acoustic lens 108E (not shown in FIG. 2). The transducer 111E converts the received ultrasound signal into a received electrical signal, which is an analog signal. The analog signal recorded by the transducer 111E is input to the receiver circuitry 112E of transceiver element 110E. The input to the receiver circuitry 112E is a sinusoidal waveform having the same frequency as the transmitted signal.

The receiver circuitry comprises a low noise amplifier (LNA) 113E, a mixer stage 114E, 115E, low pass filters 116E, 117E and analog to digital converters 118E, 119E.

The received electrical signal is amplified by the LNA 113A to obtain an amplified signal.

The amplified signal is passed from the LNA 113E to a mixer stage 114E, 115E in which the amplified signal is mixed with a single-frequency mixer signal. The single-frequency mixer signal comprises cosine and sine waveforms at the same frequency as the transmitted signal. This is an example of a homodyne architecture in which the received signal is mixed with sine and cosine waveforms having the same frequency as the transmitted signal.

The mixing with the cosine waveform in the mixer stage 114E results in an in-phase, I, signal. The mixing with the sine waveform in the mixer stage 115E results in a quadrature, Q, signal.

The result of each mixing operation is a signal having a single frequency which is double the transmitted frequency, and a DC component. Mixing a sinusoidal signal at frequency fᵢₙ with a sinusoidal signal at frequency fₘᵢₓₑᵣ will produce a signal with frequencies fᵢₙ±fₘᵢₓₑᵣ. In this case, the received signal with frequency fₖ is mixed with a mixer signal at the same frequency fₖ, thus fₖ±fₖ=2*fₖ, 0 (DC). Results of the mixing are denoted as I and Q in FIG. 2.

Only the DC components are used in subsequent processing. Thus, the signals obtained from mixing are filtered via a low-pass stage 116E, 117E. The in-phase signal is passed from the mixer stage 114E to a low-pass filter 116E which removes high-frequency components. The quadrature signal is passed from the mixer stage 115E to a low-pass filter 117E which removes high-frequency components. In the embodiment of FIG. 2, the low-pass stage 116E, 117E has a cut-off frequency of 100 kHz. In other embodiments, any suitable cut-off frequency may be used. After low-pass filtering, the SFCW architecture collects only the two DC terms, for example by sampling and holding their values.

Apart from the initial transient, the filtered signals do not show temporal dynamics. In the embodiment of FIG. 2, the filtered signals are sampled with a very low rate ADC 118E, 119E. For example, a sampling rate of 128 kHz or 256 kHz or 512 kHz may be used. The filtered signals are sampled with high bit depth, for example 24 bits or higher. The low sampling rate is possible due to the filtered signal being substantially constant with time. In some circumstances, the use of a higher bit depth or a higher bit resolution may lead to higher contrast in a resulting medical image.

The filtered I signal from low-pass filter 116E is passed to ADC 118E which samples the filtered I signal at a low rate and high bit depth. ADC 118E outputs a signal Re which is a DC value. The output signal Re comprises a single value at the frequency fₖ of the transmitted signal. The output signal Re is a real part of the frequency spectrum of the signal at the input of the receiver.

FIG. 2 illustrates a plot 200 of Spectrum (Real) against frequency. The plot 200 includes a single point 201 at frequency fₖ which is the DC value Re that is output by ADC 118E.

The filtered Q signal from low-pass filter 117E is passed to ADC 119E which samples the filtered Q signal at a low rate and high bit depth. ADC 119E outputs a signal Im which is a DC value. The output signal Im comprises a single value at the frequency fₖ of the transmitted signal. The output signal Im is an imaginary part of the frequency spectrum of the signal at the input of the receiver.

FIG. 2 illustrates a plot 210 of Spectrum (Imag) against frequency. The plot 200 includes a single point 211 at frequency fₖ which is the DC value Im that is output by ADC 119E.

After digitization, the two DC values Re, Im represent the real and imaginary parts of the frequency spectrum (at fₖ) of the signal at the input of the receiver. The two DC values Re, Im are passed to the processing resource 150.

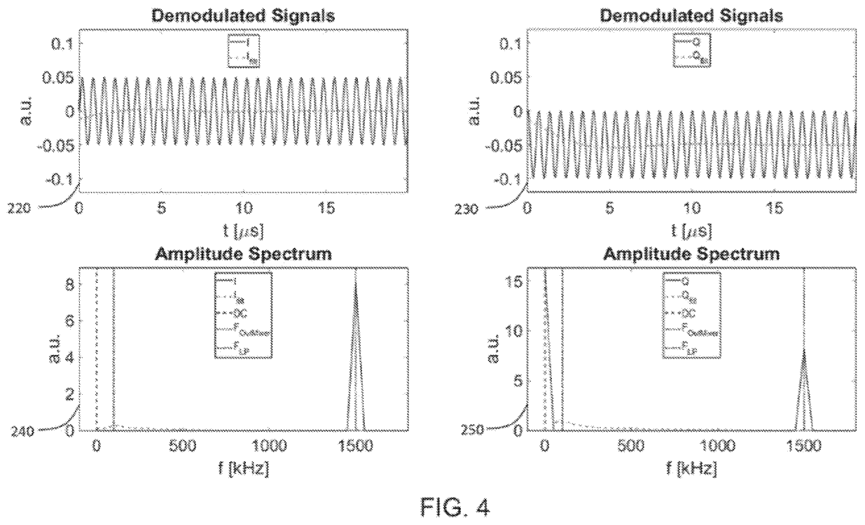

FIG. 4 shows a plurality of plots 220, 230, 240, 250. Plots 220, 230 of FIG. 4 show demodulated signals I and Q, plotted in arbitrary units versus time. It should be understood that, in FIG. 4, t=0 μs corresponds to the time of arrival of the signal at the input of the receiver. For a transmitted tone at a fixed frequency (750 kHz in the FIG. 4), the signals I and Q at the output of the mixer contain one frequency which is twice the transmitted one, and a DC term.

Plots 220, 230 show a result of filtering as a dashed line. Apart from an initial transient due to the filtering operation, the filtered signals are constant signals.

Plots 240, 250 show amplitude spectrums for I and Q which are plotted in arbitrary units against frequency, showing the received I or Q signal and a filtered version I_{filt} or Q_{filt}. A vertical line marked as DC corresponds to the DC term (zero frequency). A vertical line F_{outMixer} corresponds to the non-zero frequency at the output of the mixer A vertical line F_{LP} corresponds to a cut-off frequency of the low-pass stage.

The description of FIG. 2 above describes the processing of one single-tone continuous-wave signal. Respective DC values Re, Im are obtained for each of the single-tone continuous-wave signals in the sequence of single-tone continuous-wave signals. The processing resource 150 therefore receives from receiver circuitry 112E a respective pair of Re and Im values for each predetermined frequency used to generate the sequence of single-tone continuous-wave signals. This corresponds to one complex digital sample for each combination of receiver, transmitter and frequency. Each complex digital sample is sampled at, for example, 24 bits.

In the present embodiment, the sequence of single-tone continuous-wave signals comprises only one single-tone continuous-wave signal per frequency. In other embodiments, frequencies may be repeated such that the sequence of single-tone continuous-wave signals comprises more than one single-tone continuous-wave signal per frequency. In such embodiments, an overall Re and Im value for a frequency may be obtained by combining the Re and Im values for each single-tone continuous-wave signal in the sequence of single-tone continuous-wave signals that is transmitted at that frequency, for example by averaging. As an example, if the sequence of single-tone continuous-wave signals comprises three single-tone continuous-wave signals at a given frequency, three Re values may be averaged to obtain a combined Re value, and three Im values may be averaged to obtain a combined Im value.

Corresponding processing is performed by the receiver circuitry 112B, 112C, 112D, 112F, 112G and 112H of the other receiving elements 110B, 110C, 110D, 110F, 110G and 110H and Re and Im values for each frequency are passed to the processing resource 150 by each receiver circuitry 112B, 112C, 112D, 112F, 112G and 112H.

The controller 140 instructs the signal generator 130 to send the sequence of single-tone continuous-wave signals to each of the transceiver elements 110A, 110B . . . 110H in turn. When a transceiver element is transmitting, each of the other transceiver elements receives a signal and process said signal to obtain a respective pair of Re and Im values for each single-tone continuous-wave signal.

When each of the transceiver elements 110A, 110B . . . 110H has transmitted, the controller 140 instructs the ring-shaped array of transceiver elements 110A, 110B . . . 110H to move by a predetermined increment in a direction perpendicular to a plane of the ring-shaped array. In some circumstances, for example when operating in research mode, the increment may be set by a user. The controller 140 instructs another set of signal transmission and reception by each one of the transceiver elements as described above.

The controller 140 repeats the incremental movement of the ring-shaped array and transmission and reception of signals until a limit is reached, for example until the entire breast has been imaged or until a predetermined number of increments has been reached.

As already described above with reference to FIGS. 2 and 3, the transmitter circuit generates a square waveform, or a sinusoidal waveform known as a single tone. A single tone has programmable transmission time Tₖ, peak-peak voltage

$${V}_{Tx , pp}^{( k )} ,$$

frequency fₖ and phase (the latter is zero in FIG. 2). The transmission time may be 400 μs. In other embodiments, different transmission times may be used e.g. 200 μs or 600 μs. The peak-peak voltage may be 100 mV. In other embodiments, different peak-peak voltages may be used e.g. 1 V or 10 V. The frequencies span the range 100 kHz-1 MHz in steps of 50 kHz. In other embodiments, different frequency steps may be used e.g. 5 kHz or 100 KHz. In other embodiments, the frequencies may span the range 100 kHz-1 MHz but the frequency steps may be non-uniform in size. The phase of the single tone may be zero. In other embodiments, the phase of the single tone may be different e.g. 90 degrees or 180 degrees.

All of the previous parameters (transmission time, peak-peak voltage, frequency and phase) are independently programmable and may depend on the position along the scanning direction: e.g. a higher value of the peak-peak voltage may be employed in the regions of the breast closer to the chest, in combination with longer transmission times, to cope with increased signal attenuation due to a bigger breast volume.

As already described above, for the geometry of a single ring-array, single tones at different frequencies are transmitted sequentially by a single array element before single tones at different frequencies are transmitted sequentially by the next single array element.

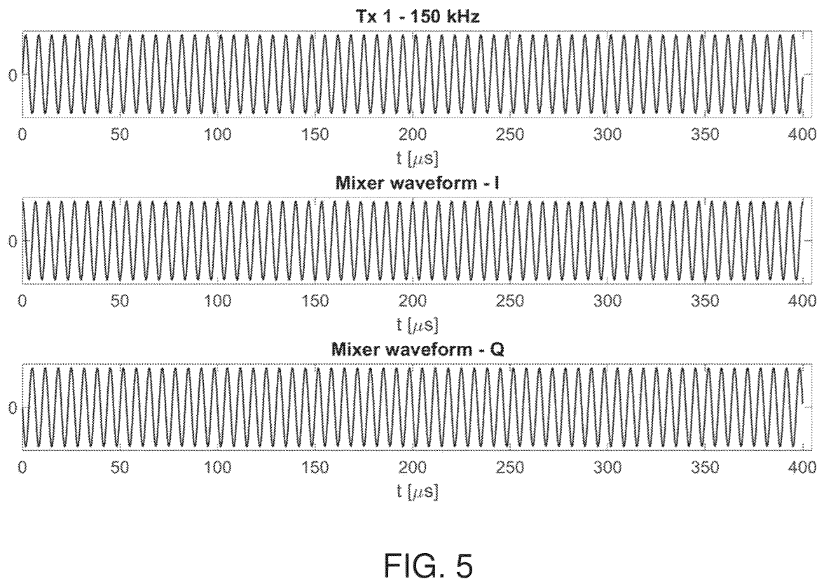

The transmission and reception of single tones will now be described with reference to FIGS. 5-12 for an ultrasound computed tomography system which is similar to that described above with reference to FIGS. 1A to 4, but which comprises an array of 512 transceiver elements arranged in a two-dimensional ring around an imaging area with array element #256 arranged opposite array element #1.

A single tone is sent to a specific array element, for example element #1. With element #1 transmitting, all the other elements receive in parallel. All the frequencies within a predetermined range are transmitted sequentially. When the transmission from element #1 is over, a single tone is sent to array element #2. All the frequencies within a predetermined range are transmitted sequentially. All the array elements transmit in turn. Only one single tone is transmitted at a time. The top plot in FIG. 5 shows an example of a single tone transmitted by element #1 at fₖ=150 kHz for a transmission time Tₖ=400 μs.

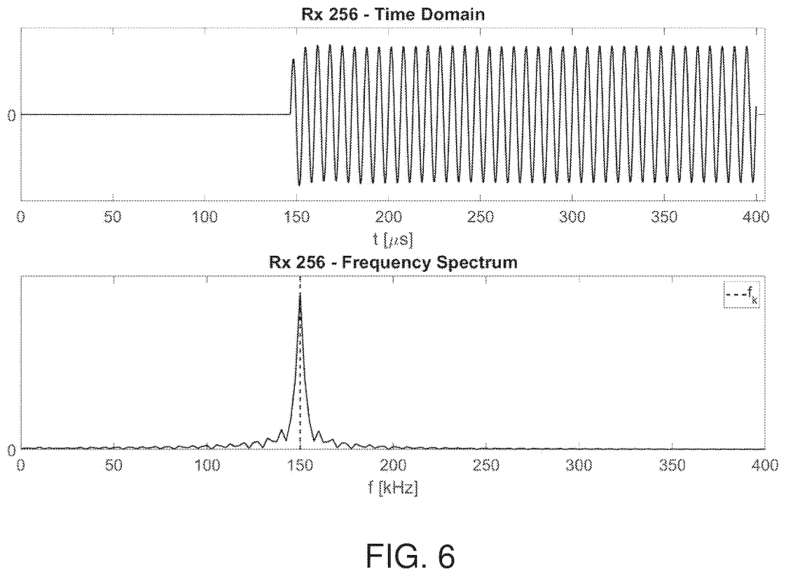

Each array element has the same receiver chain shown in FIG. 2. The first stage is a low-noise amplifier (LNA), followed by two mixers, two low-pass filters and two ADCs. Following transmission and propagation in a medium, the analog signal at the input of the LNA is a time-shifted single tone at the same frequency as the transmitted one. FIG. 6 shows such a signal for array element #256 when array element #1 is transmitting. The time of arrival may depend on the speed of sound of the insonified tissue/medium and on the known distance between the Tx/Rx pair.

The LNA may apply a gain of 20 dB (in one embodiment 10 dB, in another embodiment 40 dB, in another embodiment −10 dB etc). The LNA gains may be receiver dependent, frequency dependent and scanning-location dependent. The LNA amplification will result into a time-shifted single tone at the same frequency as the transmitted one with a bigger peak-peak voltage amplitude.

As shown in FIG. 2, the received and amplified single tone is mixed with cos and −sin waveforms (as shown in the middle and bottom plots of FIG. 5 respectively) at the same frequency as the frequency of the transmitted single tone. cos waveforms are assigned to the I branch of the mixer and −sin waveforms are assigned to the Q branch of the mixer. As shown in FIG. 5, these waveforms are temporally aligned with the onset of the transmission of the single tone.

In one embodiment, the same oscillator (not shown explicitly in FIG. 2) drives both the transmitter and the receiver circuits. In this case, a sinusoidal waveform is generated and it goes through an initial power splitter. A portion of the waveform is sent to the transmitter circuit. The other portion undergoes multiple power splitter and phase shifter stages (not shown explicitly in FIG. 2): these waveforms are sent to the I and Q branches of the mixer stage at every receiver element.

As the transmission is continuous, all the array elements receive except the transmitting one e.g. array elements #2-#512 receive while array element #1 transmits.

The peak-peak voltage of the mixer waveforms may have a value of 1 mV (in one embodiment 100 μV, in another embodiment 10 mV etc). These peak-peak values may depend on the transmitted frequency, the receiving element and the scanning location. The peak-peak voltage of the mixer waveforms,

$${v}_{Rx , pp}^{( I ; k )} and {v}_{Rx , pp}^{( Q ; k )} ,$$

may be different even for the same receiving element: e.g.

$${V}_{Rx , pp}^{( I ; k )}$$

may be 1 mV and

$${V}_{Rx , pp}^{( Q ; k )}$$

may be 5 mV etc.

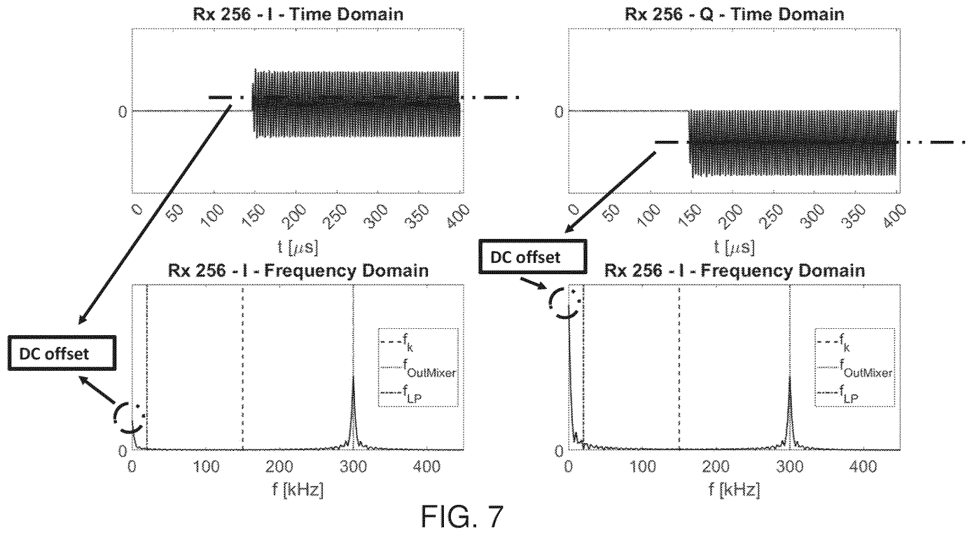

The mixer stage combines the two sinusoidal mixer waveforms shown in the middle and bottom plots of FIG. 5 with the time-shifted, LNA-amplified single tone shown in FIG. 6, all having the same frequency fₖ. The result of this operation is a time-shifted sinusoidal waveform with an output frequency f_{OutMixer} twice the transmitted frequency fₖ, and a (generally) non-zero DC component. Continuing with our example of element #1 transmitting at a frequency fₖ=150 kHz, the frequency of the signal at the output of the mixer will be f_{OutMixer}=2*fₖ=300 kHz, both for the I branch and the Q branch. FIG. 7 shows an example of I and Q waveforms for array element #256. Both waveforms exhibit a non-zero DC offset (aka DC bias), visible both in the time-domain and in the frequency-domain. It should be understood that the time t=0 μs in FIG. 7 corresponds to the onset of transmission and that the time of arrival in FIG. 7 for array element #256 is around t=150 μs.

The receiver chain samples the DC component for the I branch and the Q branch respectively, at all the receiving elements. This is accomplished by low-pass filtering the I and Q waveforms and by digitizing the steady values of these low-pass filtered analog signals.

As shown in FIG. 7, the low-pass filters may have a cut-off frequency of 20 KHz. In other embodiments the low-pass filters may have a different cut-off frequency e.g. 5 kHz or 100 KHz. The cut-off frequencies may depend on the frequency of the transmitted single tone, the receiving element and the scanning location.

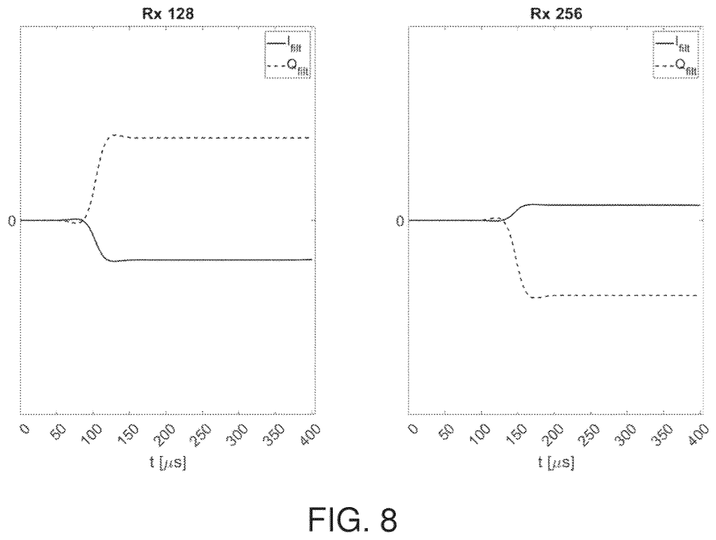

The low-pass filtered signals I_{filt} and Q_{filt} reach their respective steady values after the time of arrival at any given receiving array element. For example, FIG. 8 shows the evolution of the low-pass filtered signals I_{filt} and Q_{filt} for array elements #128 and #256 when array element #1 is transmitting. Two steady values per array element are digitized by the last stage of the receiver chain, the ADCs.

The ADC may have a sampling frequency of 128 kHz. In other embodiments the ADC may have a different sampling frequency e.g. 256 kHz or 512 kHz. The ADC resolution may be 24 bits. In other embodiments the ADC may have a different resolution e.g. 14 bits, 16 bits or 32 bits. The ADC sampling frequency may be programmable and may depend on the frequency of transmitted single tone.

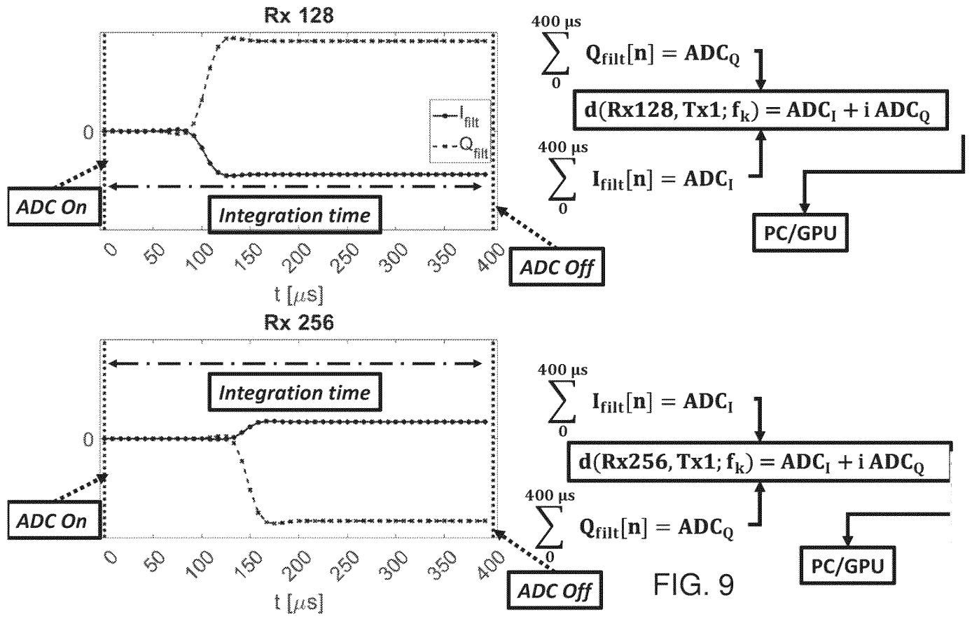

As shown in FIG. 9, the ADC may sample from the onset of the transmission, for the entire transmission time. The FPGA driving the ADC sums the digital samples saved in the ADC memory buffer: the result of this operation is two real digital samples, ADC, and ADC_{Q}, per Tx/Rx pair, at a given frequency fₖ. These two samples are combined into the complex, digital sample d(Rx, Tx; fₖ)=ADC_{I}+i ADC_{Q}, where i is the imaginary unit. The real part ADC_{I} is the result of the digitization along the I branch, and the imaginary part ADC_{Q} is the result of the digitization along the Q branch (FIGS. 2 and 9). This complex digital sample is transferred to the PC/GPU for further processing. In this embodiment, the integration times at all receiving elements are all 400 μs and are equal to the transmission time of 400 μs shown in FIG. 5. It should be understood that the real digital samples ADC and ADC, described above with reference to FIGS. 5 to 9 are equivalent to the digital samples Re and Im described above with reference to FIGS. 2 to 4.

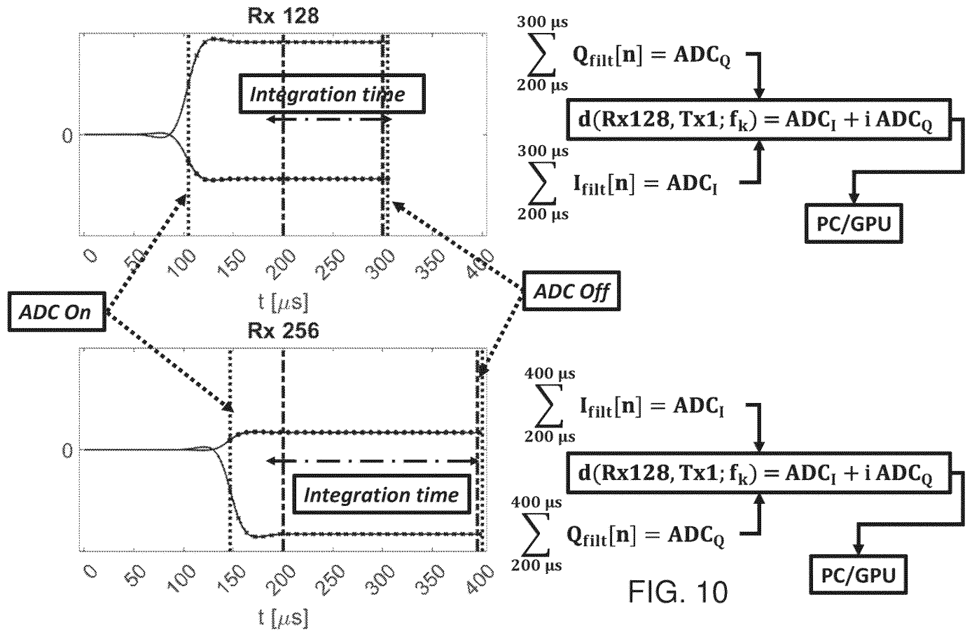

In another embodiment shown in FIG. 10, the ADC starts sampling at a specific time of arrival, until the end of the transmission time. The time of arrival may be programmable and may depend on the receiving element. The time of arrival can be easily estimated from the known distance between a Tx/Rx pair and assuming a known value for the speed of sound of the medium (for soft tissue a reference value of 1540 m/s may be used). For example, for the geometry of a single ring array with 512 elements, with element #1 transmitting, a lateral element (e.g. element #128) may start sampling around 100 μs after the onset of the transmission; an opposite element (e.g. element #256) may start sampling around 150 μs after the onset of the transmission. As above, the ADC samples saved into the corresponding memory buffers are summed by the FPGA driving the ADC: the result of this operation is a complex digital sample per Tx/Rx pair (at a given frequency). This complex digital sample is transferred to the PC/GPU for further processing. In this embodiment, the integration times may depend on the transmitted frequency, the receiving element and the scanning location. In FIG. 10, array element #128 integrates for 100 μs (from 200 μs after the onset of transmission up to 300 μs after the onset of transmission), whereas array element #256 integrates for 200 μs (from 200 μs after the onset of transmission up to 400 μs after the onset of transmission). This strategy may lead to more accurate samples, as it discards the digital samples acquired during the transition from a zero value to the steady value.

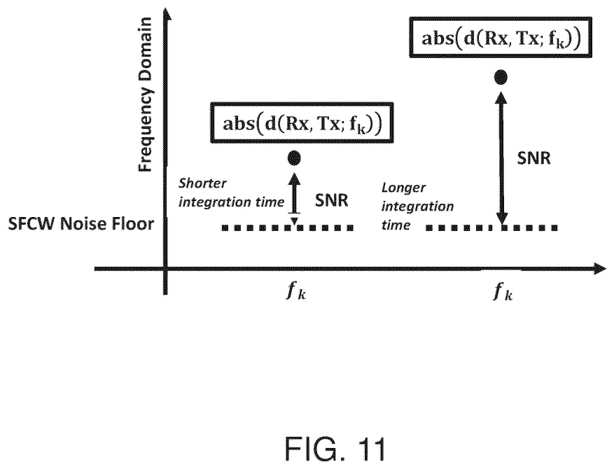

A longer integration time may result in a higher SNR and may be beneficial especially at high frequencies (as higher frequencies sounds waves are attenuated more) and for thicker/bigger breast regions (as the sound wave signal is attenuated more). In FIG. 11, it is shown that, for a given frequency, and a fixed Tx/Rx pair, the SNR is higher for a longer integration time. The SNR is here defined as the height in the frequency domain between the signal level (measured by the absolute value of the complex sample d(Rx, Tx; fₖ)) and the noise floor.

The transmission time must be as long as the desired longest integration time. The integration times may depend on the frequency of the transmitted tone and the receiving element. The transmission time may also depend on the frequency of the transmitted single tone and its peak-peak voltage.

All the array elements transmit and receive, and all the combinations are recorded. As the transmission is continuous, all the array elements receive except for the transmitting one. Thus, the sample d(Rx=Tx, Tx; fₖ) is never collected. For the geometry of a single ring-array with 512 elements, it may be convenient to store a single frequency dataset with a size of 512 (Rx)×512 (Tx) complex samples, digitized at the ADC resolution, with the implicit assumption that the samples d(Rx=Tx, Tx; fₖ) are filled with zeros. The samples d(Rx=Tx, Tx; fₖ) are not used anyway in the image formation algorithm as tomographic imaging exploits transmission data. If N_{f} frequencies are transmitted and recorded, the dataset has the size of 512 (Rx)×512 (Tx)×N_{f} complex samples, digitized at the ADC resolution.

When all combinations of Tx, Rx and frequencies are recorded at a single scanning location, the ring-array translates vertically along the z direction. The step in elevation along the z direction may be programmable and may be 5 mm. In other embodiments, different steps in elevation may be used e.g. 3 mm or 10 mm.

If N_{scans} is the number of scanning locations along elevation, a single breast dataset has the size of 512 (Rx)×512 (Tx)×N_{f}×N_{scans} complex samples, digitized at the ADC resolution.

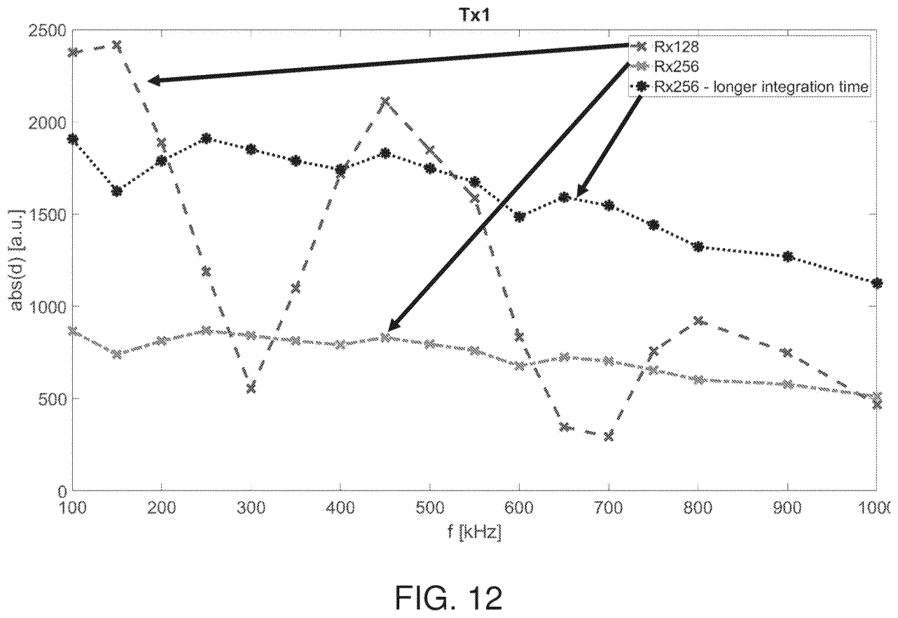

FIG. 12 shows the absolute value of complex sample d(Rx, Tx; fₖ) measured by a lateral receiver (Rx128) and by an opposite receiver (Rx256) when array element #1 is transmitting. FIG. 12 shows frequencies transmitted between 100 kHz and 800 kHz (with a step of 50 kHz), at 900 kHz and 1 MHz. The data may have been acquired by using the same integration time and by transmitting the same value for the peak-peak voltage

$${V}_{Tx , pp}^{( k )}$$

at all frequencies. As shown in FIG. 12, due to wave propagation and attenuation, the signal level at an opposite receiver may be less than the signal level at a lateral receiver. As tomography is enabled by transmission data (at lateral or opposite receivers), it is important to increase the signal level (and thus the SNR) at the receiving elements relevant for tomographic imaging, potentially by having a uniform signal level. This can be accomplished in the SFCW-USCT architecture by simply adjusting the integration times of the receiving array elements to a longer value to thereby increase the SNR.

From the foregoing discussion, one of ordinary skill in the art will understand that the respective integration times for the receiver elements are less than or equal to the transmission time for the transmitter element.

The processing resource 150 uses all of the d(Rx, Tx; fₖ) values to perform a computed transmission tomography calculation to image the medium, which in this embodiment is the breast. In the present embodiment, for each position of the ring-shaped array in the direction perpendicular to the plane of the array, the processing resource performs a computed transmission tomography calculation using the d(Rx, Tx; fₖ) values obtained at that position to obtain a two-dimensional image for that position. The processing resources then combines two-dimensional images for all positions to obtain a three-dimensional volume. In other embodiments, any suitable method of computed tomography calculation may be used.

The computed transmission tomography calculation is performed in the frequency domain.

The process of mapping ADC raw data into an image in the Cartesian space is called image reconstruction, or image formation. In medical imaging, the displayed image shows one, or more, or a combination of physical and chemical quantities responsible for image contrast. In ultrasound imaging, the contrast is given by the acoustic impedance of the medium. The acoustic impedance is the product of two physical parameters: the mass density and the speed of sound. Tissues may have different values of mass density and speed of sound, in particular the values of the speed of sound show a significant spread with respect to differences in mass density. Hence, reconstructing images that show the speed of sound of different tissues may inform clinical diagnosis. In one embodiment, image reconstruction is performed with iterative algorithms solving a numerical optimization problem. The latter is defined by a cost function C(θ):

C(θ)=∥d_{syn}(θ)−dₘₑₐₛ∥²

The cost function measures the difference (for example in the 12 norm) between measured data and synthetic data generated according to some numerical rule. The vector θ is an unknown quantity (the would-be images): solving the numerical optimization problem is equivalent to minimize the cost function, i.e. estimate the vector θ=θ₁, θ₂, . . . in correspondence of which the cost function reaches its minimum/minima. In the case of a cost function depending on a single unknown quantity θ₁, one may write:

$$\text{?} = arg {min}_{{θ}_{1}} {‖ {d}_{syn} ( {θ}_{1} ) - {d}_{meas} ‖}^{2}$$

where the hatted variable [symbol] represents the best estimate for the unknown quantity θ₁. This estimate can be accomplished via well-known iterative techniques, based for example on a simple gradient descent algorithm. This assumes that an initial guess for the unknown variable is available, [symbol] ⁽⁰⁾. Further/better estimates are obtained by iterating the initial value along the descent direction:

$${\text{?}}^{( k k + 1 )} = {\text{?}}^{( k k )} - α {∇}_{{θ}_{1}}^{( k k )}$$

where

$${∇}_{{θ}_{1}}^{( k k )}$$

is the numerical gradient of the cost function with respect to the unknown variable θ₁, at iteration number kk. The parameter α is the step-size. Thus, an estimate [symbol] can be obtained if an initial guess is available and if the gradient of the cost function can be computed. The step-size can be defined by the user. The estimated [symbol] is then displayed as a 2D image.

In one embodiment, image reconstruction is performed in the frequency domain, with iterative techniques based on a physical model (model-based image reconstruction). These assume a physical model and iteratively minimize the difference between measured data and synthetic data generated via a pre-selected model. The physical model is often encoded by a partial differential equation (PDE) describing known fundamental physics. The cost function C(θ) can be written as:

C(θ)=Σ_{Tx}Σ_{Rx}Σ_{f}_{ₖ}∥d_{syn}(Rx,Tx;fₖ;θ)−dₘₑₐₛ(Rx,Tx;fₖ)∥²

The previous sums are over all possible combinations of transmitting and receiving elements, and over all acquired discrete frequencies. In our invention, the term dₘₑₐₛ(Rx, Tx; fₖ) coincides with the complex sample d(Rx, Tx; fₖ) described above. The term d_{syn}(Rx, Tx; fₖ; θ) represents synthetic data generated with a numerical model at frequency fₖ, and for a given combination of Tx/Rx array elements. The variable θ is generic vector θ₁, θ₂, . . . where each θᵢ is a parameter describing a physical quantity. In the case in which the synthetic data are generated via a physical/parametric model described by a PDE, the numerical optimization problem is an example of a pde-constrained optimization problem. In fact, in this case, the synthetic data must satisfy a constraint given by the relevant PDE, that we can write as M(θ)=0 (where M stands for model, and the vector θ represents the physical parameters of this model). It is well known that the propagation of sound wave in soft tissue is well described by the acoustic wave equation in the time-domain and by the Helmholtz equation in the frequency domain. The latter is the single-frequency (f) PDE:

$${∇}^{2} p ( \vec{x} ; f ) + \frac{{( 2 π f )}^{2}}{{c}^{2} ( \vec{x} )} p ( \vec{x} ; f ) - s ( \vec{x} ; f ) = 0$$

where p(\vec{x}; f) is the pressure measured at location \vec{x}, c(\vec{x}) is the (generally) spatially-varying speed of sound of the medium and s(\vec{x}; f) is the source excitation (in the frequency domain) at location (\vec{x}), at frequency f (measured in Hz). ∇² is the standard Laplacian operator; mass density is ignored. In this case, the physical model M(θ)=0 is described by the Helmholtz equation, and the model parameter is the scalar quantity θ=θ₁=c, the speed of sound in m/s. In this framework, after discretizing the Helmholtz equation on a numerical grid, the physical pressure p(\vec{x}; f) coincides with the synthetic data d_{syn}(Rx, Tx; fₖ; θ=c) for a given pair of Tx/Rx array elements, at a given frequency f=fₖ, and for a given estimate of the underlying speed of sound. The iterative estimation of the speed of sound can then be written as:

$${\hat{c}}^{( k k + 1 )} = {\hat{c}}^{( k k )} - α {∇}_{c}^{( k k )}$$

As above, an initial guess has to be available, ĉ⁽⁰⁾, and a rule to compute the numerical gradient with respect to the speed of sound. Here below we describe our approach to multi-bandwidth reconstruction.

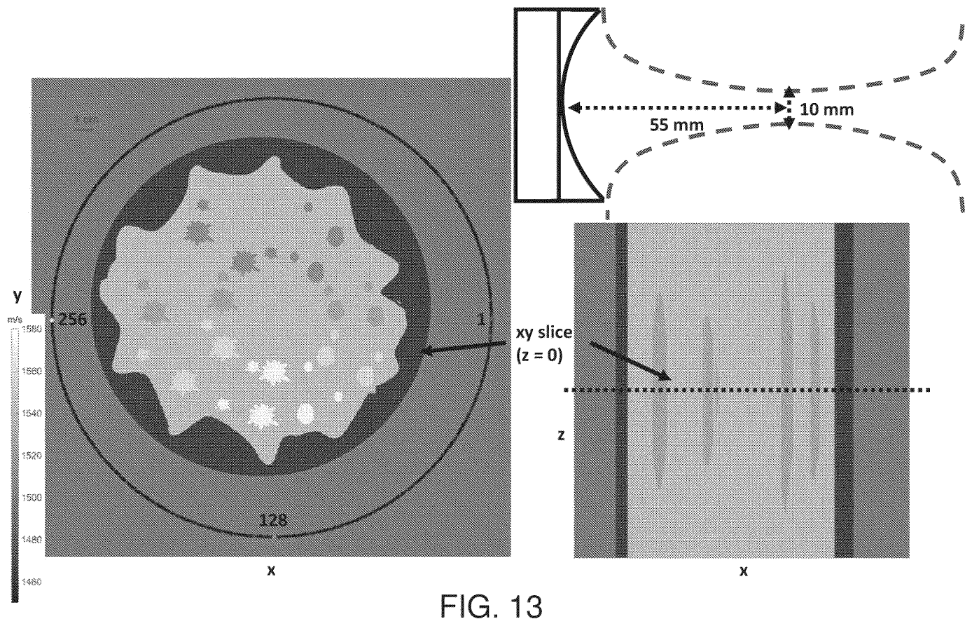

Continuing with our example of a 512 array elements ring-array, we consider the case where data is recorded for the case of a phantom sample shown in FIG. 13 at the following 13 discrete frequencies: 100 kHz, 200 kHz, 250 kHz, 300 kHz, 350 kHz, 400 kHz, 450 kHz, 500 kHz, 600 kHz, 700 kHz, 800 kHz, 900 kHz, 1 MHz. Data is generated in 3D assuming a radius of 110 mm, a focal depth of 55 mm and a beamwidth of 1 cm at 1 MHz, on a grid of size 1200×1200×68 pixels with a pixel of 0.2 mm. A single array element is drawn with a rectangular shape with a focusing acoustic lens on it. The phantom sample includes multiple generally spherical inclusions, between 5 mm and 10 mm in size. The inclusions with irregular borders may be representative of malignant masses (spiculations), the ones with regular borders may be representative of benign masses or cysts. The inclusions have been generated with variable speed of sound, thus showing different contrast with respect to the background tissue. Finally, texture has been added to mimic an actual medium. Specially, random values have been added on a pixel by-pixel base to mimic an actual medium.

The acquired frequencies are grouped in five frequency bandwidths: each frequency bandwidth is used to estimate finer details for the speed of sound, on smaller pixel sizes. Specifically, we group the frequencies as follows: [100 kHz, 200 kHz, 250 kHz-1 mm], [300 kHz, 350 kHz-0.8 mm], [400 kHz, 450 kHz-0.6 mm], [500 kHz, 600 kHz, 700 KHz-0.4 mm], [800 kHz, 900 kHz, 1000 kHz-0.32 mm].

To illustrate the basic block of the imaging algorithm, we describe in detail what are the steps to reconstruct an image in the frequency bandwidth 100 kHz, 200 kHz, 250 KHz; the measured dataset in this frequency bandwidth is a complex vector of size 512 (Rx)×512 (Tx)×3. We consider a constant initial guess, also known as a flat initial guess (constant initial velocity model). This is a 2D image of size 280×280 pixels, with a pixel size of 1 mm. The initial value of the speed of sound may be 1500 m/s (speed of sound in water). The Helmholtz equation is solved at three discrete frequencies and the corresponding synthetic data are computed. The cost function is evaluated by taking the difference between measured data and the synthetic data. The gradient is computed and the speed of sound is updated. The process is repeated for as many iterations as desired. These steps are summarized below:
I. Choose Frequency bandwidth (for example 100 kHz, 200 kHz and 250 kHz)
II. Choose pixel size (for example 1 mm)
III. Choose size of image/numerical grid (for example 280×280 pixels)
IV. Choose initial guess (for example constant image 280×280, filled with data values of 1500 m/s).
V. Start iteration loop.
VI. Solve the Helmholtz equation with the current estimate of the speed of sound (geometry of Tx/Rx is known), for all elements, with one element transmitting at the time.
VII. Extract the pressure at the receivers (geometry of Rx is known).
VIII. Compute residuals between synthetic data and measured data.
IX. Compute gradient by solving adjoint problem.
X. Update speed of sound.
XI. Repeat steps V-X for as many iterations as desired.

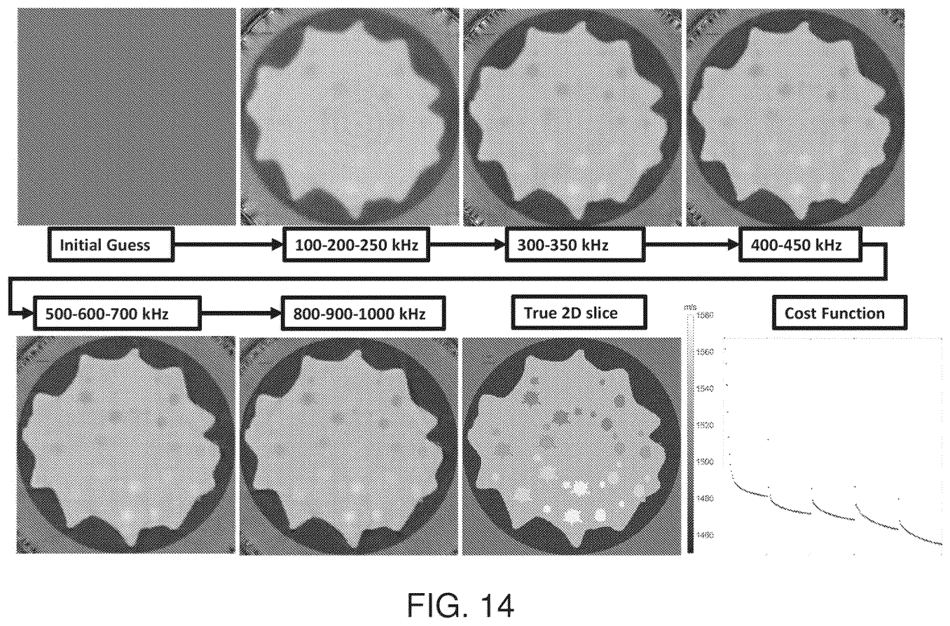

An example of a reconstructed speed of sound image in the 100-200-250 kHz bandwidth after 50 iterations is shown in FIG. 14. Better estimates for the speed of sound image are obtained in the frequency bandwidth 300-350 kHz, on a finer grid of 300×300 pixels with a pixel size of 0.8 mm. The previous estimate for the speed of sound image is first interpolated from a grid of size 280×280 (1 mm) to a finer grid of size 300×300 (0.8 mm), and the same steps are repeated, again for 50 iterations. The last estimate is stored and it is shown in FIG. 14. The principle of the multi-bandwidth reconstruction is then straightforward: the last estimate of the speed of sound image becomes the initial guess for the reconstruction on a finer grid (after being interpolated). For example, the speed of sound image may be interpolated from a grid of size 300×300 (0.8 mm) to a finer grid of size 468×468 (0.6 mm), and the image is reconstructed in the 400-450 kHz bandwidth, and so on. The result of the multi-bandwidth reconstruction is shown in FIG. 14. To avoid inverse crime, images have been reconstructed up to the maximum frequency 1 MHz, but on a bigger pixel size (0.32 mm) with respect to the actual pixel size (0.2 mm) of the true numerical phantom sample. All images are shown on the same scale 1450-1580 m/s. The improvement in image quality from low frequencies to high frequencies is evident, as expected from first principles (higher frequency=higher resolution=higher image quality). The final image shows degraded quality with respect to the true numerical phantom. This is due to the fact a ring-array is 2D system, whereas wave propagation is a 3D physical phenomenon. The out-of-plane scattering may degrade image quality in any 2D system (regardless of how the transmitter and the receiver are implemented). In particular, image quality in the xy imaging plane may reach a plateaux at the frequency bandwidth in corresponding of the (smallest) beam-width of the device. In fact, the nominal beam-width (in this example 1 cm) is reached at 1 MHz and further improvements of the speed of sound in the xy imaging plane may not be feasible. Finally, the cost function is graphed in FIG. 14 for a total of 250 iterations, with 50 iterations per frequency bandwidth. FIG. 14 shows a clear decreasing behaviour within each frequency bandwidth. This suggests that the numerical optimization problem has been solved and that the SFCW-USCT architecture can reliably produce images of increasing quality by progressively inverting for higher frequencies. In the absence of noise, all inclusions have been detected, although with difference accuracy, as expected from variable contrast, size and borders, and from general considerations of 2D reconstruction from 3D data.

The reconstruction method described above allows for great flexibility, for example the reconstruction parameters in a given frequency bandwidth may change. Specifically: the number of frequencies per bandwidth can be chosen (minimum one frequency per bandwidth), the maximum frequency determines the size of the grid and the pixel size according to which numerical scheme is employed to solve the Helmholtz equation, the step size and the number of iterations can be chosen (and the numerical optimization algorithm as well, e.g. gradient descent, conjugate gradient etc), a subset of transmitters and receivers may be chosen. All the previous parameters are independently programmable and may depend on the scanning position and the clinical task. The interpolation step on a smaller pixel size may be achieved with any interpolation algorithm, no specific interpolation algorithm is implied here.

With reference to our previous example of a planar ring-array, images will be formed at each scanning location. A 3D body part (for example a breast) will be reconstructed by stacking 2D images on top of each other. These 2D images/slices are separated by a distance equal to sampling step in elevation.

The image reconstruction algorithm runs on a dataset at a fixed scanning location (single slice reconstruction). Multiple scanning locations can be reconstructed in parallel and independently.

As already described above, tomographic images can be determined with standard iterative techniques within the general framework of model-based image reconstruction in the frequency domain. The computed tomography calculation comprises an iterative algorithm based on the numerical optimization of a cost function. The cost function C measures the difference between data recorded at a single frequency d(fₖ; Tx, Rx) and synthetic data M(fₖ; θ₁, . . . , θₙ; Tx, Rx):

$$C = {\sum }_{k = 1}^{{N}_{fSteps}} {\sum }_{T x} {\sum }_{R x} {‖ M ( {f}_{k} ; θ ; T x , R x ) - d ( {f}_{k} ; T x , Rx ) ‖}^{2}$$

where d(fₖ; Tx, Rx) is equal to d(Rx, Tx; fₖ) as defined above i.e. d(fₖ; Tx, Rx) is the complex digital sample whose real and imaginary parts are respectively equal to the digitized values of the I and Q analog signals after low-pass filtering, and optionally also integration, for a given pair of transmitter and receiver elements Tx, Rx as described above with reference to FIGS. 2 to 10, and where Real (d) is denoted as Re above and Imag(d) is denoted as Im above. The sum is performed over all possible transmitter and receiver combinations with the (implicit) assumption that tomographic imaging is enabled by transmission data. It should be understood that M(fₖ; θ₁, . . . , θₙ; Tx, Rx) is described above as d_{syn}(Rx, Tx; fₖ; θ). M(fₖ; θ) is a parametric model described by a partial differential equation (pde), where θ₁, . . . , θₙ are the physical variables in the model (e.g. the speed of sound). The estimation \hat{θ} of the physical parameters is equivalent to the minimization of the cost function of a pde-constrained optimization problem:

$$\hat{θ} = arg {min}_{θ} {‖ M ( {f}_{k} ; {θ}_{1} , … , {θ}_{n} ; T x , R x ) - d ‖}^{2}$$

subject to the physical constraint M(θ)=0. Synthetic data can be obtained with one or more physical models (dependent on the medical application): for example, the acoustic wave equation in the frequency domain (Helmholtz equation) with or without visco-acoustic terms, the elastic wave equation in the frequency domain with or without visco-elastic terms. These techniques may lead to estimates for the physical parameters θⱼ (e.g. compressional and/or shear speed of sound, acoustic and/or elastic attenuation etc). The cost function can be minimized by iteratively updating the estimate for the physical quantity of interest until convergence is reached. Once the gradient is known, a simple gradient descent algorithm with fixed step-size a can be used to iteratively update the physical quantity of interest:

$${θ}_{j}^{( i t e r + 1 )} = {θ}_{j}^{( i t e r )} - α {∇}_{{θ}_{j}}^{( i t e r )}$$

where

$${∇}_{{θ}_{j}}^{( i t e r )} = \partial C / \partial {θ}_{j}$$

is the gradient of the cost function with respect to the physical quantity of interest θⱼ at each iteration step. The calculation of the numerical gradient can be achieved by the well-known techniques of the adjoint state method. Additionally or alternatively, other optimization methods may be employed, for example gradient descent with a line search or conjugate gradient. The initial physical models (initial guesses

$${θ}_{j}^{( i t e r = 0 )} )$$

for the optimization problem may be flat/constant models. In some embodiments, first iterations are run in a low frequency regime; an updated models will then be fed to the iterative reconstitution algorithms in a higher frequency regime. The SNR of the low-frequency data and the higher-frequency data may be ensured by tuning the amplitudes and the integration times of the single-frequency tones (and their steps). Low frequency and high-frequency iterative reconstruction algorithms may be different and employ different physical models and different numerical methods to solve the wave equations (for example a partial differential equation describing a specific physical model may be solved with standard finite difference methods in one regime and spectral or finite elements methods in another regime). Regularization terms may be added to the cost function, for example to account for experimental noise and/or to improve convergence and/or smoothness of the final image.

The processing resource 150 may output a two- or three-dimensional image, for example to a display screen (not shown). The outputting may comprise any suitable image rendering, for example rendering of 2D slices or of a 3D image using any suitable projection.

The method described above with reference to FIGS. 1A to 14 may provide a safe and operator-independent method of ultrasound imaging of the breast for the diagnosis of breast cancer. The method of ultrasound imaging may be suitable for use in screening, for example in mass screening for breast cancer.

Transmission is continuous, and data are collected directly in the frequency domain rather than in the time domain. No time domain data is collected. Only frequency domain data is collected after digitization. Data collection in the frequency domain is combined with image reconstruction methods that are also in the frequency domain.

By transmitting sequentially at different frequencies (with programmable steps) and collecting the two DC values for each transmission, a desired bandwidth may be synthesized.

Unlike conventional hand-held ultrasound probes that exploit only pulse-echo data, the SFCW-USCT system uses SFCW transmission data i.e. the SFCW-USCT system is not a pulse-echo system. The SFCW-USCT system may additionally use SFCW diffraction data, which may also be referred to as lateral data.

Control of single tones is provided. Single tone amplitudes, frequencies, phases, transmission times and integration times can be controlled independently. A stepped-frequency continuous-wave ultrasound computed tomography architecture may be designed to have any desired SNR per frequency bin/step.

In embodiments described above, the array of ultrasound elements is a two-dimensional array which is moved in a direction perpendicular to a plane of the two-dimensional array. In some embodiments, the array of ultrasound elements is a three-dimensional array rather than a two-dimensional array. The three-dimensional array of ultrasound elements may comprise a plurality of two-dimensional arrays, for example a stack of multiple two-dimensional ring arrays. In some such embodiments, the three-dimensional array is configured to image the entire medium, for example the entire breast, without moving. In other embodiments, the three-dimensional array moved as described above.

The system of FIG. 1A and FIG. 1B comprises a planar ring array, in which all elements act as transceivers, and with an acoustic lens to focus the signal into the imaging plane. Data is acquired by a single circular ring. The breast is scanned vertically for two-dimensional reconstruction. Reconstruction is performed slice by slice. Physical quantities θⱼ may be estimated as 2D images and a 3D volume formed by stacking 2D images.

Some other geometries are inherently 3D, i.e. physical quantities θ; may be estimated as 3D volumes co-located with the device and a full 3D volume formed by stacking thin 3D volumes.

In the embodiment of FIG. 1A and FIG. 1B, all transducer elements have the same focusing properties, for example the same thickness, height and width. Additionally, the same acoustic lens is used for each element, so that the focus onto the imaging plane is the same at all locations in z. One single ring scans the breast vertically and collects data sequentially in elevation/along z.

In other embodiments, stepped-frequency continuous-wave signals and processing may be used with any suitable geometry. In some embodiments, the acoustic lenses may be omitted. The elements may comprise at least part of at least one circular ring, or any other suitable array shape.

From the foregoing description of the SFCW-USCT system, one of skill in the art will understand that: the transmitted signal is a single tone at a programmable frequency; the received signal at the input of the LNA is a time-shifted single tone at the same frequency as the transmitted one; the analog signals after amplification undergo a homodyne receiver; the analog signals at the input of the ADC are DC signals; data are sampled in the frequency domain; and data are processed via a model-based iterative technique reconstructing a physical parameter, for example the speed of sound.

It may be understood that the SFCW-USCT system and methods have been described above purely by way of example, and that modifications may be made to the SFCW-USCT system and methods without departing from scope of the invention as defined according to the appended claims. For example, although the SFCW-USCT system 100 was described above as having 8, 512 or 2048 array elements, the SFCW-USCT system may have more or fewer array elements. Regardless of the number of array elements, at any one time, one of the array elements may transmit, whilst the other array elements receive.

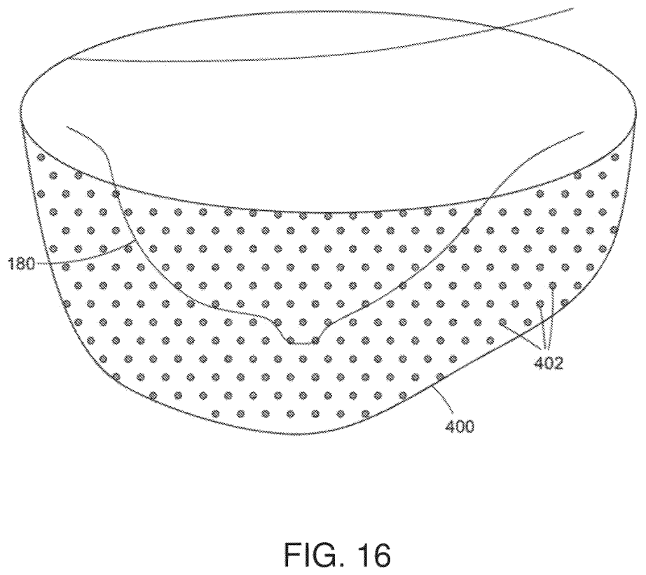

FIG. 16 illustrates an alternative geometry for an SFCW-USCT system in which ultrasound elements 402 are arranged on an inside surface of a bowl 400. An array of elements 402 in FIG. 16 may be described a bowl array, surface array or matrix array. The elements 204 are uniformly distributed in the inside of the bowl and directed towards the breast 180. In the embodiment of FIG. 16, the breast 180 is sensed in three dimensions and reconstructed using a three-dimensional reconstruction. No mechanical motion of the array is used.

In the embodiment of FIG. 16, all transducer elements have the same focusing properties, for example height and width. In other embodiments, different transducer elements may have different focusing properties. In further embodiments, elements may be arranged on the inside of any convex surface which may have any suitable shape.

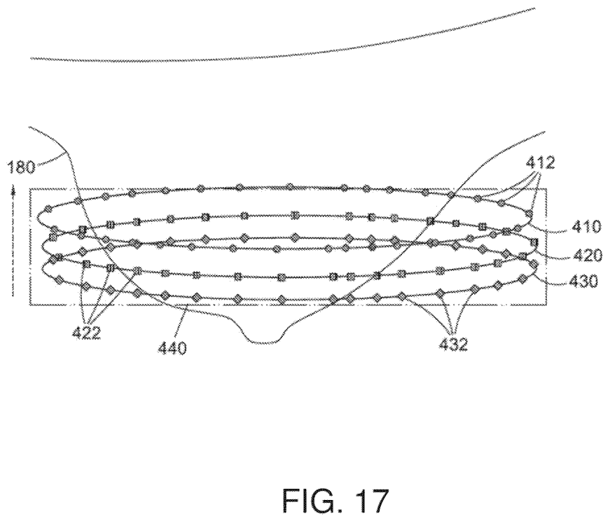

FIG. 17 illustrates a further alternative geometry for an SFCW-USCT system in which transducer elements 412, 422, 432 are arranged in three circular rings 410, 420, 430. All transducer elements 412, 422, 432 transmit and receive regardless of the ring they are part of. When a transducer element from a given ring 410 transmits, other transducer elements 412, 422, 432 from all three rings 410, 420, 430 receive.

In some embodiments, the three circular rings 410, 420, 430 may have different radii. For example, in order to follow breast curvature a bottom ring 430 (positioned further from the breastbone) may have a smaller radius, a middle ring 420 may have an intermediate radius and a top ring 410 may have a bigger radius. In the embodiment of FIG. 17, the breast 180 is scanned vertically for 3D reconstruction. The three rings move as a single mechanical entity. Data are collected sequentially in elevation/along z. A 3D volume is reconstructed by stacking thin 3D volumes co-located with the three rings (illustrated as dashed rectangle/field of view 440).

In other elements, a different number of rings may be used.

In the embodiment of FIG. 17, all transducer elements belonging to the same ring have the same focusing properties. In some embodiments, ultrasound elements of the different rings may have different acoustic properties. For example, elements of different rings may have different foci, beamwidths, frequency bandwidths and/or size of single transducer elements. Transducer elements on different rings may transmit at different frequencies. In some embodiments, a different transducer spacing is used on different rings.

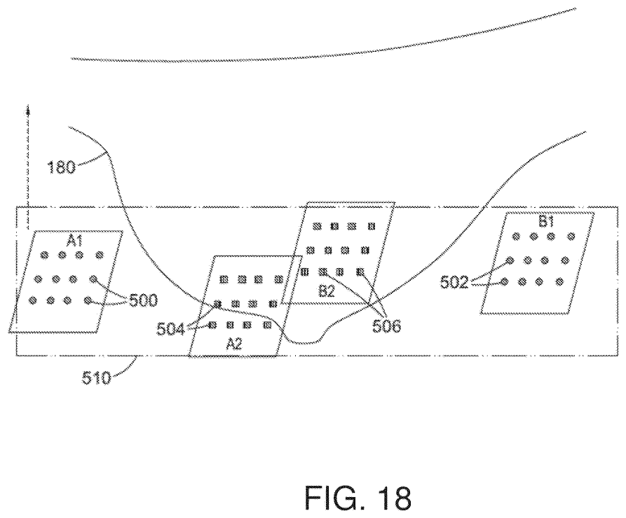

FIG. 18 illustrates an embodiment in which transducers are arranged in two groups of opposite transducer arrays, each having multiple rows. In the embodiment shown in FIG. 18, a first group, Group 1, includes a first two-dimensional array A1 having three rows of transducer elements 500 on a first side of the breast 180, and a second two-dimensional array B1 having three rows of transducer elements 502 on a second, opposite side of the breast 180. Transducer elements in array A1 or array B1 may lie on a flat surface or on a curved or bowl-shaped surface. Arrays A1 and B1 rotate synchronously around the breast 180 such that they are always at opposing sides of the breast 180.

A second group, Group 2, has a similar geometry but different acoustic properties. The second group includes a third two-dimensional array A2 having three rows of transducer elements 504 on a third side of the breast 180, and a fourth two-dimensional array B2 having three rows of transducer elements 506 on a fourth side of the breast 180 which is opposite to the third side. Transducer elements in array A2 or array B2 may lie on a flat surface or on a curved or bowl-shaped surface. Arrays A2 and B2 rotate synchronously around the breast 180 such that they are always at opposing sides of the breast 180.

In the embodiment of FIG. 18, each pair of arrays is positioned such that signals transmitted from a first array are transmitted through the medium and received by a second array that is positioned directly opposite to the first array. In other embodiments, when the first array is positioned at a first side of the medium, the second array may be positioned at any appropriate position around the medium, which may not be directly opposite the first array. For example, the second array may receive signals laterally from the first array.

Elements of Group 1 may have first characteristics and elements of Group 2 may have second, different characteristics. For example, elements of Group 1 may have different focusing properties from elements of Group 2. Transducer elements from Group 1 and Group 2 may transmit and receive at different frequency bandwidths. Group 2 may have different acoustic properties (for example, height, width) from Group 2.

The use of different acoustic properties by Group 1 and Group 2 may allow data to be obtained in a lower frequency range (for example 200 kHz to 300 kHz) by Group 1 and data to be obtained in a higher frequency range (for example 2 MHz to 3 MHz) by Group 2. The different acoustic properties may allow a high signal-to-noise ratio to be obtained both at lower frequencies and at higher frequencies. Both the lower frequency data and the higher frequency data may be used in image reconstruction.

Mechanical rotation is used to surround completely the breast. Data are collected sequentially in elevation/along z. Vertical scanning is used for 3D reconstruction (stack of thin 3D volumes). In some embodiments, distances between array A1 and array B1 and between array A2 and B2 may be controlled electronically and/or mechanically while scanning the breast in elevation. At a fixed location in elevation, data are used to reconstruct the thin volume co located with the two groups (illustrated as dashed rectangle/field of view 510).

In other embodiments, any suitable number of arrays may be used.

In the SFCW-USCT system geometries described above with reference to FIGS. 1A and 1B, 16, 17 and 18, all transducer elements act both as transmitters and receivers (with the only rule that the transmitting element is not receiving during transmission). In other embodiments, separate transmitters and receivers may be used.

Additionally, the system may work in different modes, for example screening mode, high resolution mode, research mode, follow-up treatment mode etc. The design of the final geometry may include an imaging sub-system transmitting at lower frequencies (with an SNR that allows for the accurate estimation of initial velocity models, that are critical in iterative reconstruction methods) and another imaging sub-system transmitting at higher frequencies (that enables high-resolution details to be recovered via further iterations, potentially for clinically-dependent tasks). Consider, for example, the geometry of FIG. 18. In some embodiments, Group 1 may be used to perform low-frequency imaging, for example for screening. A combination of Group 1 and Group 2 may be used to obtain higher-resolution data for research or for follow-up.

In some embodiments, transmitters and receivers have different focusing properties. In some embodiments, a transmitter can be a group of elements. A group of elements may transmit together for transmission of a plane wave. In some embodiments, a receiver is a group of elements.

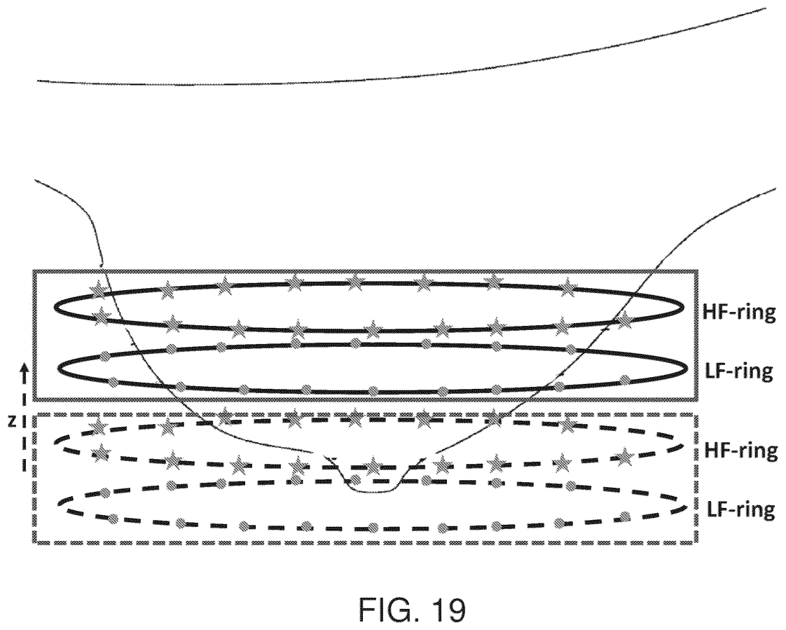

In another SFCW-USCT system geometry shown in FIG. 19, two rings may be present. These two rings may have different number of elements, different radii and different acoustic focusing properties. In particular, the array elements in the low-frequency (LF) ring transmit and receive in the 100 kHz-500 kHz bandwidth, whereas the array elements in the high-frequency (HF) ring transmit and receive in the 500 KHz-1000 kHz bandwidth. For this geometry, one may have two independent transmitting channels, i.e. one element from the LF-ring and one element from the HF-ring may transmit simultaneously, e.g. at 100 KHz and 600 KHz respectively. In receiving, the LF-ring elements record the signal due to the 100 kHz transmission and the HF-ring elements record the signal due to the 600 KHz transmission. In terms of the imaging parameters, these two rings behave as two completely independent units. The two rings are separated by a fixed distance and scan the breast via mechanical motion. Slices at the same elevation are co-registered by the two rings; the two rings may allow for a low-frequency image and a high frequency one. The LF-ring elements may be used to perform low-frequency imaging, for example for screening. A combination of the LF-ring elements and the HF-ring elements may be used to obtain higher-resolution data for research or for follow-up.

It should be understood that the breast, or other body parts, may be immersed in a water-bath (a tank filled with water). The water bath represents the coupling medium for ultrasound wave propagation.

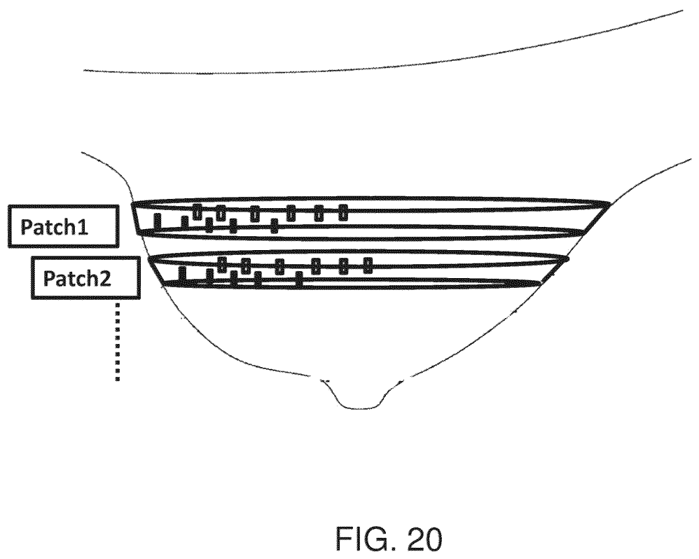

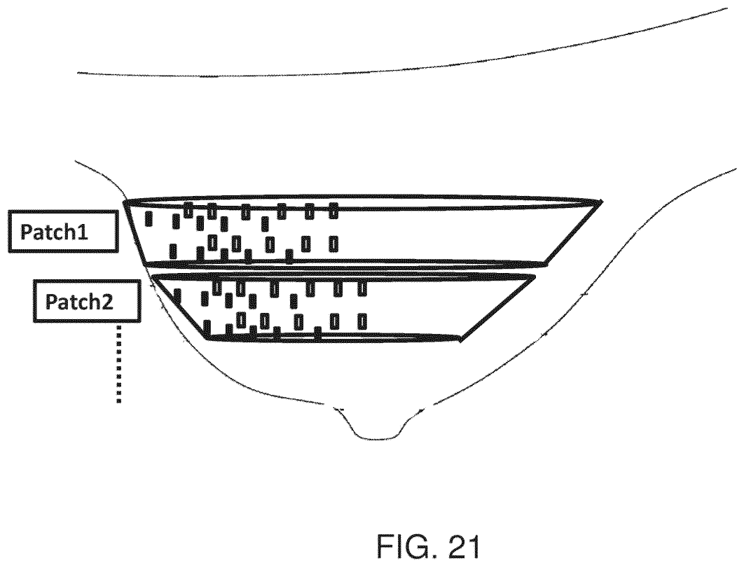

In another embodiment, the sensing/imaging device may be accomplished with a wearable patch. One of the disadvantages of a wearable patch is that the emitted power is low, as the driving circuit is on the patch itself. Hence, a wearable patch with pulsed transmission may not be able to record transmission data, because it is low-powered (usually a few Volts). On the other hand, a continuous transmission with a peak-peak voltage of a few hundreds of mV and a long enough integration time may be able to capture transmission data. Wearable patches may enable 2D imaging. For example, FIG. 20 shows a linear array sitting on a circular stripe applied on the breast for 2D imaging. Alternatively, wearable patches may enable 3D imaging. For example, FIG. 21 shows a matrix array sitting on a foil wrapped around the breast for 3D imaging. A potential drawback of some of the geometries described above is that the focus in elevation is fixed; this is because an acoustic lens has a fixed focal depth and a fixed beam width at a given frequency. As breasts may show significant variability in size, a device with a given radius and a fixed focus in elevation may be suitable only for some breast sizes. In the case of a wearable patch, one may design wearable stripes and/or foils with different radii, different acoustic focusing, variable array elements etc, allowing for imaging which is more personalized. The wearable patches may be applied directly on the skin, with or without the application of an acoustic gel as a coupling medium. In this embodiment, wearable patches are intended to be single-use, disposable medical devices.

In the SFCW-USCT system and methods described above, the same oscillator (not shown explicitly in FIG. 2) drives both the transmitter and the receiver circuits.

In another SFCW-USCT embodiment, two different synchronised oscillators may be used: Osc₁ and Osc₂. One master clock is used to synchronise both oscillators Osc₁ and Osc₂. Osc₁ may drive the transmitter circuit: a sinusoidal waveform is generated and sent to the transmitter circuit. Osc₂ may drive the receiver circuits: a sinusoidal waveform is generated and undergoes multiple power splitter and phase shifter stages: these waveforms are sent to the I and to the Q branch of the mixer stage at every receiver element. In this embodiment, the transmitter and the receiver circuits are decoupled.

In a further SFCW-USCT embodiment, multiple synchronised oscillators may be used: Osc₁ and Oscₖ where k is the number of receiver elements. One master clock is used to synchronise all of the oscillators Osc₁ and Oscₖ. Osc₁ drives the transmitter circuit: a sinusoidal waveform is generated and sent to the transmitter circuit. Each Oscₖ drives a different receiver circuit: each Oscₖ generates a sinusoidal waveform and this undergoes a single power splitter and phase shifter stage before being sent to the I and the Q branch of the mixer stage of a corresponding single receiver element. In this embodiment, the transmitter circuit, the receiver circuit and the mixer waveforms are fully decoupled.

In another embodiment, the multi-bandwidth image reconstruction may be complemented by a crosstalk-free multi-bandwidth reconstruction with phase-encoding. In the conventional multi-bandwidth reconstruction, the Helmholtz equation (or an equivalent pde describing some physical model) is solved transmitter by transmitter, under the hypothesis that only one array element transmits at the time. In the embodiment with phase-encoding, the Helmholtz equation is solved under the hypothesis that all array elements transmit together (as far as it concerns synthetic data). When this happens, the gradient computation suffers from the cross-talk noise. In order to remove or reduce this cross-talk noise, a phase-encoding vector is generated at each iteration step. The phase encoding vector is generated frequency-by-frequency and for each array element. The synthetic data are generated by solving the Helmholtz equation with all array elements transmitting together, each encoded by a phase-encoding term. The measured data are encoded by the same phase-encoding terms; the residuals at the receivers and the gradient are then computed. The speed of sound is updated and a new iteration can start with a different encoding vector. In this embodiment, the phase-encoding term is a pseudo-random vector drawn a from pseudo-random uniform distribution in the interval [0, 2π]. In order for the phase-encoding principle to work (i.e. reduce the cross-talk noise), this pseudo-random vector has to be generated at each iteration step. To guarantee reproducibility, this pseudo-random vector has to be generated in a deterministic way. This can be accomplished by fixing the seed of the pseudo-random algorithm in a deterministic way (for example equal to the iteration number), and then use known pseudo-random algorithms generating uniform numbers in the interval [0, 2π] with the chosen seed. These steps are summarized below:
I. Choose frequency bandwidth (for example 100 kHz, 200 kHz and 250 kHz)
II. Choose pixel size (for example 1 mm)
III. Choose size of image/numerical grid (for example 280×280 pixels)
IV. Choose initial guess (for example constant image 280×280, filled with 1500).
V. Start iteration loop.
VI. Fix seed, for example equal to the iteration number. Draw pseudo-random numbers from a pseudo-random uniform distribution in [0, 2π]. Encode source terms with pseudo-random phase term. Sum encoded sources.
VII. Solve the Helmholtz equation with the current estimate of the speed of sound (geometry of Tx/Rx is known), and with encoded sources.
VIII. Extract the pressure at the receivers (geometry of Rx is known).
IX. Encoded measured data with the same pseudo-random phase terms. Sum encoded measured data.
X. Compute residuals between encoded synthetic data and encoded measured data.
XI. Compute gradient by solving adjoint problem.
XII. Update speed of sound.
XIII. Repeat steps V-XII for as many iterations as desired.

In other words, after choosing the number of iterations, the number of array elements and the discrete frequencies, the encoding vector can be thought of as a look-up table that is generated only once, even before measuring the data. It is important to stress that in the crosstalk-free multi-bandwidth reconstruction with phase-encoding, the way of acquiring data does not change. Single tones are transmitted sequentially, frequency by frequency, element by element. In the reconstruction process, the synthetic data mimic the situation where all the array elements transmit at once, and all the array elements receive together. In the reconstruction process, the acquired data are encoded with a sum (as if they had been recorded with all the array elements transmitting and receiving at once, case which is impossible to realize for a continuous transmission). The phase-encoding strategy may be employed to estimate a velocity model starting from a constant initial guess, and the standard multi-bandwidth reconstruction may further refine it.

In another embodiment, the physical model of the Helmholtz equation allows for a complex speed of sound. It is known that the propagation of sound waves with energy absorption can be described by the same Helmholtz equation above where the speed of sound is allowed to be complex, i.e. c=cᵣₑₐₗ+i c_{imag}. The real part of the speed of sound cᵣₑₐₗ represents the velocity at which waves propagate in a medium, the complex part c_{imag} represents frequency attenuation. In this case, the cost function depends on two parameters, θ₁=cᵣₑₐₗ and θ₂=c_{imag}. Both parameters can be updated within the same iteration step by simply replacing the expression of the gradient with the same expression without taking the real part. In this case, the initial guess has to be a complex vector. Reconstructing the frequency attenuation in addition to the real part of the speed of sound may be clinically relevant, as different tissues may have similar values of the (real part of the) speed of sound (hence they may not show sufficient image contrast), but they may attenuate frequencies differently (hence the imaginary part of the speed of sound may show image contrast). It's important to stress that this distinction exists only in the physical model employed for the reconstruction; no change is needed to the way SFCW-USCT data are captured. In this embodiment, two independent images are displayed (per slice/ring location), one representing the real part of the speed of sound the other representing the imaginary part of the speed of sound.

In another embodiment, a generalized Helmholtz equation may be employed. In this case, there exist two concepts for the speed of sound: the compressional c (compressional) and the shear c (shear). The generalized Helmholtz equation may allow for frequency attenuation as above, in other words

$${c}^{( compressional )} = {c}_{r e a l}^{( compressional )} + i {c}_{imag}^{( compressional )} and {c}^{( s h e a r )} = {c}_{r e a l}^{( s h e a r )} + i {c}_{imag}^{( s h e a r )} .$$

In this case, the cost function depends on four physical parameters:

$${θ}_{1} = {c}_{r e a l}^{( compressional )} , {θ}_{2} = {c}_{imag}^{( compressional )} , {θ}_{3} = {c}_{r e a l}^{( s h e a r )} and {θ}_{4} = {c}_{imag}^{( s h e a r )} .$$

In this case, the update of the four parameters may occur within the same iteration step or, as it is also common, in an alternating fashion. This embodiment may be clinically relevant where one wants to probe, for example, the musculoskeletal human apparatus: a more accurate physical model may have to include both the compressional speed of sound and the shear speed of sound. In fact, when imaging body parts that contain bones/stiff tissue, the approximation of sound waves as described by the simple Helmholtz equation only with the compressional speed of sound may not be accurate enough, as the mismatch between the physical model and the data may affect the resulting image. In this embodiment, four independent images are displayed (per slice/ring location), the first representing the real part of the compressional speed of sound, the second representing the imaginary part of the compressional speed of sound, the third representing the real part of the shear speed of sound, and the fourth representing the imaginary part of the shear speed of sound.

The previous embodiments may be run with or without phase-encoding. In fact, the phase-encoding principle is based on the linearity of the relevant pde with respect to the source term: this is true both for the Helmholtz equation (with a real or complex speed of sound) and for the generalized Helmholtz equation (with real or complex compressional and shear speeds of sound).

In another embodiment, image reconstruction is achieved through reverse-time migration in the frequency domain (RTM-FD). The images reconstructed in this way are complementary to the speed of sound images, as they also contain the information on the mass density of the underlying tissue. In other words, an RTM image is related to the acoustic impedance of the medium, thus providing additional information/contrast. An RTM image may be thought of as the tomographic equivalent of a standard B-mode reflectivity map. In this embodiment, one image is displayed (per slice/ring location).

It is implied that the different image formation methods may be employed for different clinical applications, and they may not be mutually exclusive, for example an RTM image may be displayed together to the speed of sound (real or imaginary, compressional or shear). It is worth stressing again that the same SFCW-USCT measured data may enable multiple image formation algorithms.

A SFCW-USCT system may allow for multiple imaging protocols. In a screening protocol (optimized for sensitivity, i.e. detection of masses, either benign or malignant), the imaging protocol may employ only the low frequencies of a single ring-array, or the LF-ring of the two rings system described above. The images of FIG. 14 illustrate that all inclusions (as small as 5 mm) may be detected in the 100-450 kHz bandwidth. As such, the system will have to transmit only the relevant frequency steps from 100 kHz to 450 kHz, thus accelerating acquisition times, which may be a limiting factor in a mass screening program. Also, the reconstruction times up to 450 kHz are faster, as solving the Helmholtz equation at higher frequency is more computationally intensive. In an imaging protocol optimized for specificity (i.e. classification of already detected masses as benign or malignant, in order to rule in or rule out a disease), a SFCW-USCT system may employ higher frequencies, in the bandwidth 500 kHz to 1 MHz. This could also be applicable to the case of a treatment plan and/or follow-up (for example during chemotherapy). For research applications, the imaging protocol may employ a finer frequency step and longer transmission and/or integration times (the latter not always consistent with clinical scan times); the aim could be the study of the acoustic properties of tissue, or the in-depth analysis of anatomical features of the breast or other body parts/organs/tissues.

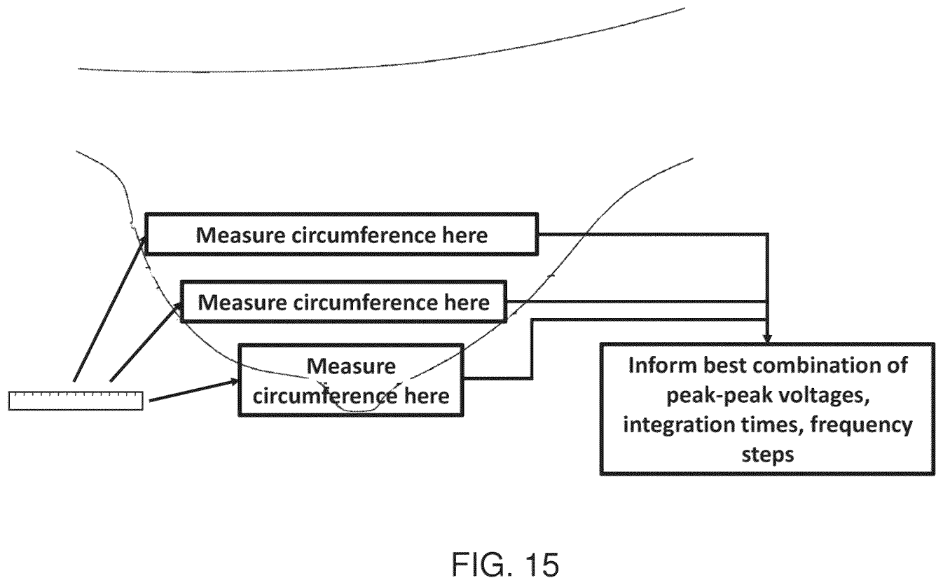

In conclusion, the SFCW-USCT architecture is more versatile, and it may allow for planning imaging protocols in advance. In addition, as illustrated in FIG. 15, a woman undergoing a breast scan may measure the circumference of the breast at three locations (nipple region, median region, chest region) prior to the breast scan. These measures may inform a suitable choice of imaging parameters (transmission time, integration time at the receivers and along the scanning direction, peak-peak voltage, frequency steps). This choice of the imaging parameters can be further tuned in combination with the woman having a dense breast, or a fatty one, or at risk of developing a disease.

The processing resource may additionally or alternatively process the Re and Im values to obtain a conventional B-mode image. Conventional B-mode images I may be obtained with a standard delay-and-sum beamforming in the frequency domain, for example via the formula

$$I ( x ) = {\sum }_{k = 1}^{{N}_{fSteps}} {\sum }_{T x} {\sum }_{R x} {W}_{T x} {W}_{R x} {\vert d ( {f}_{k} ; T x , Rx ) \vert}^{2} exp ( - 2 π {if}_{k} {τ}_{T x R x} ( x ) )$$

where |d(fₖ; Tx, Rx)|² is the power spectrum of the data collected at frequency fₖ, i.e. d(fₖ; Tx, Rx) is the complex, digital sample whose real and imaginary part are respectively equal to the digitized values of the I and Q analog signals above after low-pass filtering, and optionally also integration, for a given pair of transmitter and receiver elements Tx, Rx). Real (d) is denoted as Re above and Imag(d) is denoted as Im above. W_{Tx} and W_{Rx} are weights functions applied respectively to the transmitter and receiver apertures to control the shape and sidelobes of the beams, τ_{TxRx} collectively contains the time of flight delays from a specific transmitter Tx to a point target (pixel x) and from the point target (pixel x) to a specific receiver Rx. The first sum in the previous expression is over all the stepped frequencies, the second sum is over all transmitter and receiver combinations relevant for pulse-echo imaging, i.e. over those combinations that exploit reflection data.

Embodiments above are described with reference to ultrasound imaging of breast tissue. In other embodiments, an apparatus and/or method similar to those described above may be used for medical imaging of any suitable anatomy of any human or animal subject, which may be diagnostic imaging for any suitable clinical application. The anatomy imaged may not be the breast. The anatomy imaged may comprise any body part that can be at least partially surrounded by one or more geometries, for example the geometries of FIGS. 1A, 1B, or any of FIGS. 16 to 21. For example, the anatomy imaged may comprise a brain, a leg, an arm, a chest, a neck or one or two testicles. Multiple devices or device geometries may be designed that may scan multiple regions of the body.

The main application described so far has been breast imaging. Clinical applications to other body parts may be relevant too, for example brain, chest, neck, legs, arms, testicles.

In further embodiments, a system, an apparatus and/or method similar to those used above may be used for a non-medical application, for example, non-destructive testing, seismic exploration, oil and gas industries.

Each feature disclosed in the description and (where appropriate) the claims and drawings may be provided independently or in any appropriate combination.

Comparison of SFCW-USCT System with Hypothetical Pulsed System that Uses Direct RF Sampling

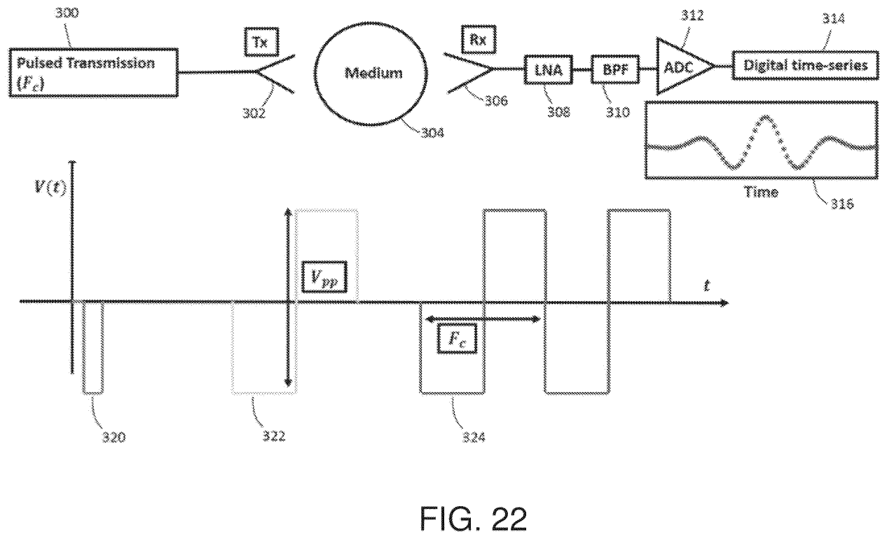

A system using a stepped-frequency continuous-wave transmission, for example as described above with reference to FIGS. 1A to 14, is now compared to a hypothetical pulsed system that uses direct RF sampling as shown in FIG. 22. It is assumed that the hypothetical pulsed system has 2048 independent transceiver elements arranged in a two-dimensional ring-shaped array around the medium to be imaged with a fixed element transmitting and the remaining elements receiving in parallel. The hypothetical pulsed system may transmit at a frequency of 3 MHz.

When using direct RF sampling, to prevent aliasing, data need to be collected at least at twice the Nyquist frequency. However, in a real device and due to noise, the sampling frequency is never equal to Nyquist, and is usually at least 4 times the central frequency. Thus, it is assumed that the system has as many ADC as the number of independent channels sampling at 12 MHz with a bit depth of no more than 14 to 16 bits.

Typical driving voltage waveforms 320, 322, 324 are shown in FIG. 22. Different possible choices for a pulsed driving voltage waveform are shown, which include a unipolar driving waveform 320, a bipolar driving waveform 322, and a driving waveform 324 including more than one cycle at central frequency F_{c}. With a single negative excitation, one usually probes the transducer impulse response: the voltage amplitude and the pulse-width must be large enough to cause the transducer element to resonate within its bandwidth. Alternatively, one can control more precisely the central frequency and the bandwidth of the transmission pulse by driving the transducer with one or more cycles at F_{c}.

The ADC are normally on as soon as the transmission is over. In order for the waves to travel through the breast and reach the opposite elements, the driving peak-to-peak voltage value may be around 100 Vpp, or more. This voltage, in combination with the pulsed nature of the system, may put constraints on the RF peak power absorbed by the transmitter circuit. Finally, one has to transmit within the limits for I_{SPTA} (spatial peak temporal average intensity) and other relevant acoustic outputs as required by the regulating agencies.

A typical direct RF sampling architecture based on a pulsed transmission is shown in FIG. 22. A pulsed transmission 300 at F_{c} is transmitted by a transducer 302 through a medium 304. The signal received by a transducer 306 is usually amplified by a LNA (low noise amplifier) 308, which is one of the first stage of the analog front-end of any receivers as input signals have too low amplitudes to be digitized properly. The signal is then band-passed by a band pass filter (BPF) 310 in the bandwidth of interest, which depends on the bandwidth of the transducer and the driving voltage waveform. Finally, the signal is sampled by an analog to digital converter (ADC) 312 and a digital time-series 314 is stored. Image reconstruction (sound of speed maps for example) can be performed in the time-domain or in the frequency domain after an FFT.

Using an RF sampling method as described above, collecting data for the entire ring array at a fixed location requires N_{Tx} transmissions, and since the system records all Tx/Rx combinations, one ends up with N_{Tx}*N_{Rx}*Nₜ real digital samples, where Nₜ is the number of digital samples stored per transmission. If we use the values N_{Tx}=N_{Rx}=2048 and Nₜ=2000 (assuming we record long enough to allow waves to travel from one transceiver to the opposite one), we will have 2048×2048×2000 (real, discrete time samples)×16 bits~16 GB per one ring location. For a breast which extends for about 10 cm from the chest and collecting data with a spatial sampling of 1 mm, one will have 100 independent scanning locations for a total amount of ~1.5 TB of data per single breast. In practice, data may be collected from both breasts.

When reconstructing breast images in the frequency domain, a limited set of frequencies could be used, for example 10 frequencies. However, in an RF sampling architecture, the use of a limited number of frequencies would not typically reduce the hardware requirements because a large amount of time domain data is collected before conversion to the frequency domain. In contrast, when data is collected in the frequency domain, the use of fewer frequencies may reduce the demands on the hardware.

The stepped frequency principle relies on the fundamental properties of the Fourier transform (analysis and synthesis). We briefly review these properties.

It is well known that the DFT (discrete Fourier transform) of a discrete time-domain signal x(n) of length N is given by the expression:

$$X ( k ) = {\sum }_{k = 1}^{N} x ( n ) exp ( - 2 π i ( k - 1 ) ( n - 1 ) / N )$$

The DFT is usually performed via the well-known algorithm of the FFT (Fast Fourier Transform). The result is a complex vector of length N. This vector has some remarkable properties, in particular the real part of the FFT is symmetric around the Nyquist frequency (F_{Nyq}=Fs/2), Re(X(2+j))=Re(X(Nfft−j)), and the imaginary part of the FFT is antisymmetric, Im(X(2+j))=−Im(X(Nfft−j)), for j≥1 (we limit ourselves to the case of a real signal). Nfft is the number of points over which the FFT operation is performed. Here, it is assumed that Nfft=N.

By Fourier analysis, one refers to the output of the DFT and the study of the spectral contents of a signal in the frequency domain. The inverse DFT (IDFT) is given by:

$$x ( n ) = \frac{1}{N} {\sum }_{k = 1}^{N} X ( k ) exp ( + 2 π i ( k - 1 ) ( n - 1 ) / N )$$

which is normally accomplished via an inverse FFT (IFFT).

Each term in the previous sum is a complex exponential at a fixed frequency. The so-called Fourier synthesis is equivalent to the previous sum, where one can perfectly reconstruct the time-domain signal by summing over all the complex exponential terms at a fixed frequency. The signal is then decomposed in Fourier components. We define a Fourier component at a fixed frequency k as the discrete, real, time domain signal:

$$F {C}_{k} ( n ) = \frac{1}{N} X ( k ) exp ( + 2 π i ( k - 1 ) ( n - 1 ) / N ) + c . c .$$

where c.c. stands for complex conjugate. A Fourier component at a fixed frequency is then a sinusoidal waveform whose amplitude is determined by the amplitude of the FFT vector at the same frequency and with a (generally) non-zero phase:

$$F {C}_{k} ( n ) = {A}_{k} sin ( \frac{2 π ( k - 1 ) ( n - 1 )}{N} + {φ}_{k} )$$

Finally, we define a (pure) tone at a fixed frequency k as a sinusoidal waveform at the same frequency with frequency dependent amplitude and zero phase ψ₀=0:

$$P {T}_{k} ( n ) = {B}_{k} ( \frac{2 π ( k - 1 ) ( n - 1 )}{N} + {ψ}_{0} )$$

In use, phase values for the single-tone continuous-wave signals may be any phase value and are not required to be zero.

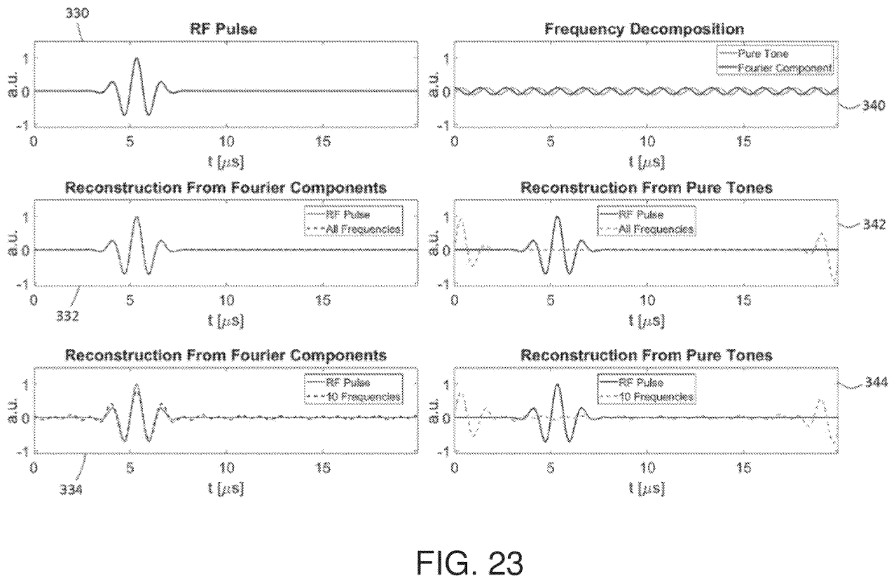

Concepts are illustrated in FIG. 23. We consider a time domain signal in the shape of an RF pulse at a central frequency F_{c}=750 KHz as shown in plot 330. A Fourier component at a fixed frequency and a pure tone at the same frequency and with the same amplitude as the corresponding Fourier component (Bₖ=Aₖ) are shown in plot 340; note the phase difference between the two. Note also the difference in peak positive/negative amplitudes for the RF pulse and the Fourier Components/pure tones. The RF power and voltage amplitudes needed for single tone transmission may be much less than an RF power and voltage amplitudes used for RF transmission. By summing over all the Fourier components (plot 332), one can reconstruct the original signal, as it should be. The sum of all the pure tones (plot 342) fails to reconstruct the signal: while the amplitudes of the tones are the same as the ones of the Fourier components, the phases do not add up (coherently) to reconstruct the signal. Plots 334 (Fourier components) and 344 (pure tones) shows the result of the Fourier synthesis by considering only a limited number of frequencies, specifically 10 frequencies around F_{c}. In this case, reconstruction by summing only over a subset of the Fourier components (plot 334) is inaccurate and one observes oscillations which do not cancel out.

As shown in FIG. 23, the accurate reconstruction of time-domain profiles requires the collection of a large number of frequencies. Collecting a limited subset of frequencies and performing reconstruction in the time domain would introduce artefacts in the image space. On the other hand, collecting only a few discrete frequencies in a bandwidth of interest and performing reconstruction in frequency space (for example, the reconstruction using the outputs of FIG. 2 as described above) may result in artefact-free images.

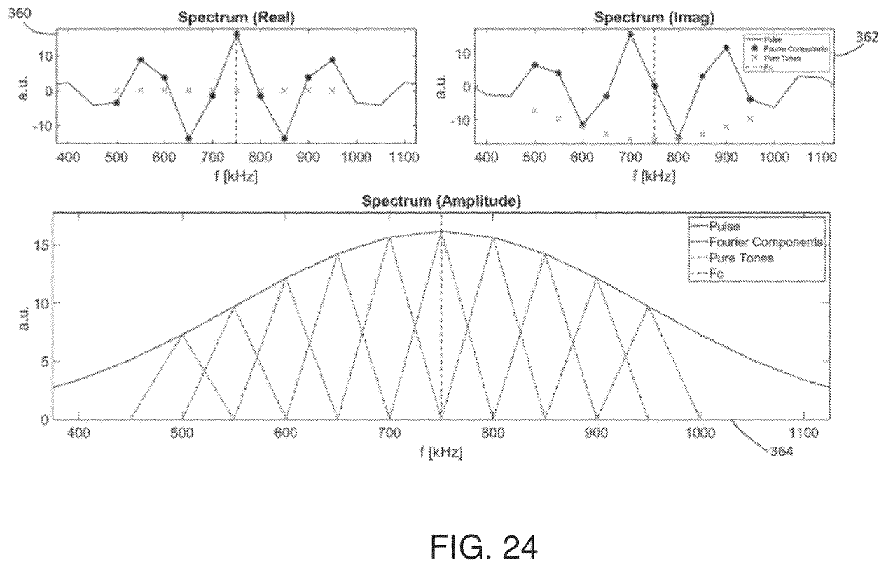

FIG. 24 shows the Fourier analysis. The real and imaginary parts of the FFT of the RF pulse are shown around the central frequency (real part in plot 360, imaginary part in plot 362). The real and imaginary parts of the FFT of each Fourier component match the values of the FFT of the RF pulse, as expected from the theory. Plot 364 shows an amplitude spectrum.

For visualization purposes, the phase spectra of the Fourier components (shown as asterisks in plots 360, 362) and of the tones (shown as crosses in plots 360, 362) only show the values in correspondence of the underlying frequency, while the amplitude spectra show the full curves for both.

As the pure tones have zero phase, the phase spectrum will not match the phase spectrum of the RF pulse. In particular, following our sign convention for the FFT (exp(−iθ)=cos(θ)−i sin(θ)) and since we have defined the pure tones as sine functions with zero phase, after projecting along the Fourier axes one expects zero real part and negative imaginary part for the frequency spectrum of each tone. However, since we have chosen equal amplitudes for the Fourier components and the pure tones, the amplitude spectrum of the RF pulse, the Fourier components and the pure tones will match.

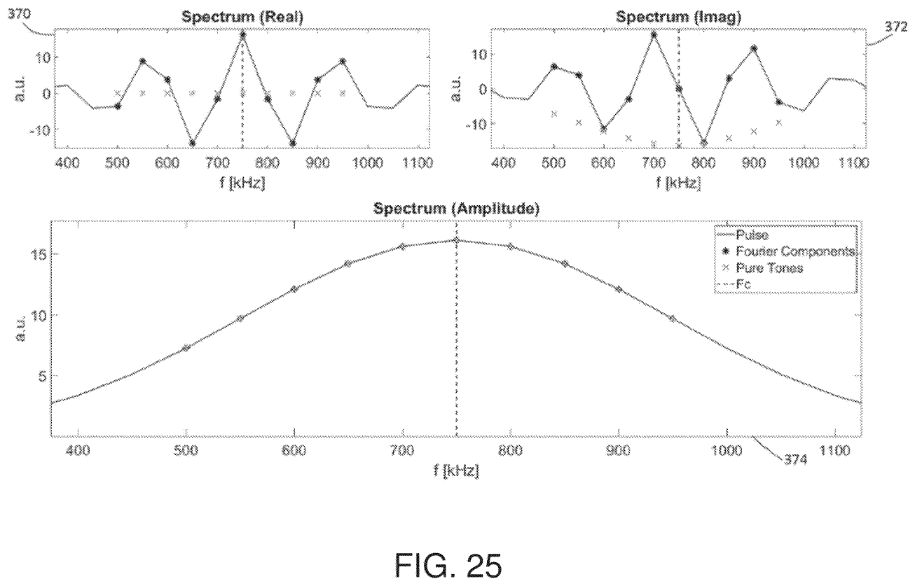

Turning again to the stepped-frequency continuous-wave architecture, by transmitting sequentially at different frequencies (with programmable steps) and collecting the two DC values for each transmission, one can synthesize a desired bandwidth. The results of said synthesizing are shown in FIG. 25.

We consider a pulsed transmission at a central frequency F_{c}. After wave propagation, the signal is received and digitized following a direct conversion architecture as shown in FIG. 22. The phase spectrum and the amplitude spectrum of the digitized signal are shown in plots 370, 372, 374.

We then consider multiple, sequential transmissions at ten different frequencies around F_{c} (pure tones), having the same amplitudes as the Fourier components of the pulsed signal. The received signal for each transmission undergoes the SCFW receiver path as shown in FIG. 2 and two DC values are stored per each transmission. The amplitude spectrum of the pure tones (shown as crosses) and of the pulsed transmission match as expected.

FIGS. 24 and 25 show the FFT spectrum of the Fourier components if we wanted to transmit them with amplitude and phase determined by the Fourier transform of the pulsed transmission and digitize them via a SCFW architecture. In this case the phase spectra overlap, which proves that the SFCW architecture is correctly extracting the frequency spectrum (amplitude and phase). The amplitude spectra of the pure tones collected with a SFCW architecture match the amplitude spectrum of an equivalent pulsed-based transmission system with direct RF sampling.

One pair of values (shown as crosses) at a fixed frequency corresponds to the Re and Im values at the ADC output in FIG. 2.

FIG. 25 shows an output of a stepped-frequency continuous-wave (SFCW) architecture: in this case one can extract directly the real and imaginary parts of the spectrum (2 real samples in the discrete case). Thus, plot 374 of FIG. 25 only shows dots corresponding to the spectrum of the tones.

Although the stepped-frequency continuous-wave architecture for ultrasound CT (SFCW-USCT) is mathematically equivalent to a pulsed-based ultrasound CT system with direct RF sampling, the SFCW-USCT system may provide superior performance (e.g. lower emitted power and lower noise floor) and may be more versatile than a pulsed-based ultrasound CT system with direct RF sampling. A geometry of a single ring array in which all of the elements act as both transmitters and receivers is assumed for the comparison.

In the pulsed-based ultrasound CT system, all transmissions are driven by the same driving voltage waveforms (single negative excitation, one or more cycles at a fixed central frequency and similar). The peak-to-peak voltage

$${V}_{p p}^{Pulsed}$$

is fixed. The transmission is pulsed, i.e. the driving voltage is different from zero over a limited amount of time. RF peak power and average power are different.

In SFCW-USCT, the driving voltage waveform is a sinusoidal one, whose frequency can be stepped within a pre-determined range. Voltage amplitude

$${V}_{pp}^{SFCW} ( k )$$

and phase ψ(k) are programmable and frequency dependent. The transmission is continuous. Since the duty cycle is 1, RF peak power and average power are equal.

In the pulsed-based ultrasound CT system, once transducer dimensions, materials, acoustic lens and driving voltage waveform are fixed, the pulse shape cannot be changed (as measured for example by a hydrophone in water). In other words, the frequency contents and the relative frequency amplitudes cannot be altered.

In SFCW-USCT, after selecting transducer dimensions, materials and acoustic lens, the frequency contents and the relative frequency amplitudes can be programmed.

In the pulsed-based ultrasound CT system, as soon as the transmission is over, all the elements (including the one that has just finished transmitting) record the received signal. This may be accomplished by having as many independent ADC as the number of physical transducer elements. The ADC are on when transmission is over.

In SFCW-USCT, during the transmission, all the elements (except the one that is transmitting) record the received signal. This may be accomplished by having as many independent ADCs as the number of physical transducer elements. The ADCs may be on from the onset of the transmission as shown in FIG. 9.

In the pulsed-based ultrasound CT system, multiple firings from the same transducer are possible with the scope of increasing the SNR. In this case, the recorded time-series from the first and the second transmission are averaged, and so on for multiple transmissions.

In SFCW-USCT, multiple continuous transmissions (with the same amplitude, frequency, phase and integration time) from the same transducer are possible with the scope of increasing the SNR. In this case, the two recorded DC values from the first and the second transmission are averaged, and so on for multiple transmissions.

In SFCW-USCT, the frequency is stepped through all the desired values. Amplitude, phase and integration time may be varied.

In the pulsed-based ultrasound CT system, after collecting data from the same transducer element (one or multiple firings), another transceiver starts transmitting, data are collected as before and the process continues until all Tx/Rx combinations have been exhausted. The temporal duration of each transmission/recording (sometimes known as the PRF, the pulse repetition frequency) is fixed and has to allow for waves to reach all the elements in the array. The time in between consecutive transmissions may be tuned and has to allow for echoes from previous transmissions to decay below the receiver noise floor.

In SFCW-USCT, after collecting data from the same transducer element (one or multiple transmissions), and after going through all the frequency steps, another transceiver starts transmitting, data are collected as before and the process continues until all Tx/Rx combinations have been explored (with the rule that all the elements receive except the transmitting one). The temporal duration of each continuous transmission may be equal to the integration time, which may also be referred to as the dwell time. The integration time may be frequency dependent and allows for waves to reach all the elements in the array. The time in between consecutive transmissions may be tuned and allows for echoes from previous transmissions to decay below the receiver noise floor.

For a single array, the device is then moved to a different location along the direction perpendicular to the chest and the process is repeated until all the locations have been scanned.

In the pulsed-based ultrasound CT system, data are digitized (at least) at 4 times the central frequency. For example, for F_{c}=3 MHz, one may have a sampling frequency of Fₛ=12 MHz. Note that even though frequency inversion is performed in the 1 MHz region, one nevertheless samples at 4×F_{c} because aliasing can compromise the lower frequencies. For every transmission one has Nₜ (real) time samples per receiver sampled (in the most favourable scenario) at 16 bits.

In SFCW-USCT, since the analog signals after mixing and low-pass are pure DC signals, one can employ a low sampling rate ADC in the kHz region and with a high bit depth, for example 24 bits. The sampling frequency and the number of bits of the ADC do not depend on the values of the transmitted frequencies: digitizing DC values for transmitted frequencies of 1 MHz and 3 MHz can be accomplished with the same ADC. For every continuous transmission at a fixed frequency, one has 2 (real) frequency samples per receiver, sampled at 24 bits. For every transmitter, one has 2 (real) frequency samples per receiver, sampled at 24 bits, times the number of frequency steps N_{fSteps}.

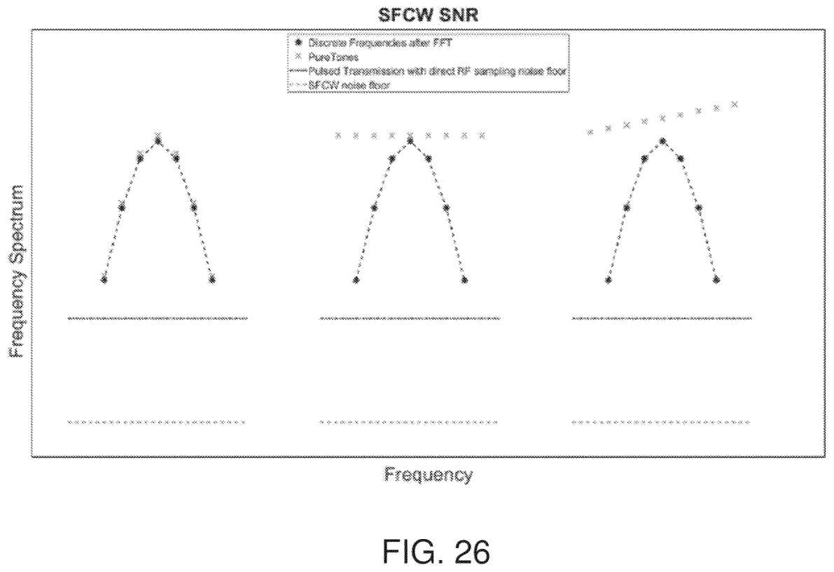

In the pulsed-based ultrasound CT system, when evaluating the SNR of the system, the signal level (shown as asterisks in FIG. 26) essentially depends on the

$${V}_{pp}^{Pulsed}$$

of the driving voltage waveform, while the noise figure of the system (shown as a point-dashed line in FIG. 26) is essentially determined by the total bandwidth of the receiver (MHz region). The dashed line connecting the asterisks in FIG. 26 is a reminder that these data are obtained after an FFT over a digital time-series.

In SFCW-USCT, when evaluating the SNR of the system, the signal level (shown as crosses in FIG. 26) depends on the

$${V}_{p p}^{S F C W} ( k )$$

of the driving voltage waveform, which can vary with the frequency, while the noise figure of the system (shown as a dashed line in FIG. 26) is essentially determined by the total bandwidth of the receiver (kHz region). The SNR per frequency (i.e. the ratio in the frequency domain between the signal level at a specific frequency and the noise floor) can be chosen by tuning the amplitudes of the different tones at different frequencies and/or by tuning integration/dwell times of single tones and thus may be much superior to the SNR of a pulsed-based USCT system.

FIG. 26 compares an SNR of a pulsed-based system with direct RF sampling with an SNR achievable with a SFCW-USCT system. Three scenarios are shown. In a scenario shown on the left of FIG. 26, the tones are transmitted with the same amplitude as the Fourier components of an equivalent RF pulsed transmission. In a scenario shown in the middle of FIG. 26, the tones are transmitted with the same amplitude as the Fourier component (at the central frequency) of an equivalent RF pulsed transmission. In a scenario shown at the right of FIG. 26, the tones are transmitted with increasing amplitudes while stepping through the frequencies, to cope, for example, for tissue attenuation and transducer bandwidth. In all cases, SNR gain and higher data fidelity are ensured.

With the SFCW-USCT architecture, one has the flexibility to program the peak-peak voltage levels of the singles tones independently, frequency-by-frequency, and to choose different integration times at different receivers, for example a longer integration time at opposite receivers. Thus, the SNR of the SFCW-USCT architecture can be shaped as desired. In effect, the SFCW-USCT architecture may enable imaging at frequencies below 400 kHz, in contrast to a pulsed/RF device which may suffer from poor SNR performance below 400 KHz and which may not therefore be capable of imaging at frequencies below 400 KHz.

The rate of detection of inclusions may be higher when using SFCW-USCT: in a clinical test, this translates into higher sensitivity. The second is that the irregular margins of the irregular inclusions (the spiculations of cancer masses in a clinical setting) are more pronounced when using SFCW-USCT, and distinguishing round inclusions from irregular ones is more robust: in a clinical test, this translates into higher specificity. To summarize, the SFCW-USCT architecture, due to its great flexibility in tuning the SNR, may allow for increased sensitivity, increased specificity and higher image quality than a pulsed device with direct RF sampling.

When compared with a pulsed device with direct RF sampling, a SFCW-USCT (stepped-frequency continuous-wave ultrasound computed tomography) architecture may allow greater flexibility in collecting data in the frequency domain. The SFCW-USCT system may have much bigger SNR and higher data fidelity, lower storage needs and/or much lower demands for the absorbed RF peak power for the transmitter circuit. Within the tomographic setting, the SFCW architecture may provide improved SNR and data fidelity.

An SFCW system may have lower peak-to-peak voltage than a pulsed system. An SFCW system may have lower absorbed RF power than a pulsed system.

Use of a higher number of bits may translate into higher acoustic contrast in final images, for example 24 bits or more for SFCW-USCT vs 14 bits for pulsed/RF. This also means better data fidelity.

A SFCW system may have lower storage needs that a pulsed system, for example a few Gb per breast vs 1 Tb per breast with a pulsed/RF system.

Since the SNR of SFCW-USCT system is expected to much bigger and one can program the frequency steps and the voltage amplitudes, in some circumstances one may need fewer frequencies for the inversion in order to achieve an image of similar or better quality with respect to a pulsed USCT system. In some embodiments, a different geometry may be used than in a pulsed USCT system. In some embodiments, a lower number of transmitters and/or receivers may be used than in a pulsed USCT system.

Control of single tones is provided. Single tone amplitudes, frequencies, phases, transmission times and integration times can be controlled independently. A stepped-frequency continuous-wave ultrasound computed tomography architecture may be designed to have any desired SNR per frequency bin/step. Such a precise control of individual frequency bins, amplitudes and steps cannot be provided in a pulsed system.

The SFCW-USCT systems and methods described may be used to obtain an artefact-free image in the 100 kHz-350 kHz bandwidth, starting from a constant/flat initial guess, without inverse crime. For breast tissue (whose speed of sound values are widely known, in the range 1450-1580 m/s), the solution disclosed here shows that a robust image may be obtained already at low frequencies with the SFCW-USCT architecture.

In the pulsed-based ultrasound CT system, if image reconstruction is performed in the frequency domain, one needs to take the FFT of all Tx/Rx combinations (after a potential digital filtering operation on the raw data) and extract the discrete frequencies of interest. The frequency bin/resolution is determined by the sampling frequency of the ADC (which is fixed) and by the number Nₜ of digitized time samples (which is also fixed).

In SFCW-USCT, since data are collected in the frequency domain (with programmable frequency steps), the system is naturally designed for performing inversion in the frequency domain. No additional operations on the data are needed.

Control of single tones is important. For a pulsed transmission, the pulse cannot be easily shaped, and the relative frequency contents and the SNR per frequency follow accordingly. If one transmits a few cycles at F_{c}=400 kHz, the power spectrum at 1 MHz will be lower than the corresponding value at 400 kHz. Since one expects an increased resolution at higher frequencies, one may wish to have a bigger SNR at higher frequencies (compatible with computing costs). If one transmits a few cycles at 1 MHz, the SNR in the 400 kHz is poor and thus the quality of initial velocity models at lower frequencies can affect reconstruction at higher frequencies.

The SFCW-USCT architecture may be designed to have any desired SNR per frequency bin/step. This is not possible with a pulsed USCT system, regardless of how the receiver path is implemented.

The main application described so far has been breast imaging. Clinical applications to other body parts may be relevant too, for example brain, chest, neck, legs, arms, testicles. The chest and the brain may be more challenging to image due to their bigger size and the presence of bones/stiff tissue that introduce higher acoustic mismatch. Due to increased size, increased body complexity, signal attenuation and wave scattering, in order to have a signal level above noise floor for transmission data, the peak-peak voltage of a pulsed transmission may be much higher than 100 Vₚₚ. This may have many drawbacks. First, medical transducers commonly employed for diagnostic imaging may not stand such high values of pulsing voltage: in this situation, array elements would crack quickly. Second, a very high pulsing voltage may violate the safety guidelines for diagnostic imaging when it comes to parameters like the mechanical index, the I_{SPTA} etc. Third, the signal level for transmission data may be captured with very low number of bits, as the noise floor of a receiver sampling in the MHz regions is generally higher than the noise floor of a receiver in the kHz region (as in our invention). Taken together, a pulsed architecture with direct RF sampling may not be feasible at all to collect transmission data. Thanks to its flexibility in SNR due to combinations of peak-peak voltages, integration times and frequency steps, a SFCW-USCT architecture may achieve more penetration with respect to a conventional pulsed architecture with direct RF sampling, and it then may allow ultrasound tomographic imaging for body parts that are not accessible with a pulsed/RF scheme.

1-29. (canceled)

30. An ultrasound computed tomography method comprising:
transmitting through a medium, by a transmitter positioned on a first side of the medium, a sequence of single-tone continuous-wave signals, the sequence of single-tone continuous-wave signals comprising at least one single-tone continuous-wave signal having a first frequency and at least one single-tone continuous-wave signal having a second, different frequency;
receiving, by each of a plurality of receivers positioned at an opposite side of the medium, the sequence of single-tone continuous-wave signals that has been transmitted through the medium;
for each of the plurality of receivers, mixing each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and
processing the obtained in-phase and quadrature values by computed transmission tomography to obtain an image of the medium.

31. The method according to claim 30, wherein the medium comprises breast tissue.

32. The method according to claim 30, wherein the method further comprises:
moving the transmitter and receivers by a succession of positional increments relative to the medium and perpendicular to a plane of the transmitter and receivers; and
for each of the positional increments, performing a respective further transmission of the sequence of single-tone continuous-wave signals and processing respective in-phase and quadrature values obtained from said further transmission by computed transmission tomography to obtain a respective two-dimensional image; and
wherein the obtaining of the image of the medium comprises combining said two-dimensional images to obtain a three-dimensional volume.

33. The method according to claim 30, wherein the transmitter and the receivers form part of an array of ultrasound elements arranged around an imaging area in which the medium is positioned.

34. The method according to claim 33, wherein the array is a circular or part-circular array.

35. The method according to claim 33, wherein the array is a three-dimensional array comprising a plurality of two-dimensional arrays, optionally wherein the two-dimensional arrays are circular or part-circular, further optionally wherein the two-dimensional arrays are of different sizes.

36. The method according to claim 35, wherein ultrasound elements of different two-dimensional arrays have different characteristics.

37. The method according to claim 33, wherein the array is arranged on a convex surface, for example a bowl-shaped surface.

38. The method according to claim 33, wherein the array comprises at least one pair of opposing two-dimensional arrays, and wherein the method further comprises rotating the at least one pair of opposing two-dimensional arrays around the medium, optionally wherein the at least one pair of opposing two-dimensional arrays comprises a first pair of opposing two-dimensional arrays comprising ultrasound elements having first characteristics and a second pair of opposing two-dimensional arrays comprising ultrasound elements having second, different characteristics.

39. The method according to claim 30, wherein the first frequency is between 100 kHz and 20 MHz and the second frequency is between 100 kHz and 20 MHz.

40. The method according to claim 30, wherein the sequence of single-tone continuous-wave signals comprises single-tone continuous-wave signals having a pre-programmed set of frequencies.

41. The method according to claim 30, wherein the sequence of single-tone continuous-wave signals comprises two or more single-tone continuous-wave signals having the first frequency, and wherein the method further comprises combining the in-phase values for said two or more single-tone continuous-wave signals to obtain a combined in-phase value for the first frequency and combining the quadrature values for said two or more single-tone continuous-wave signals to obtain a combined quadrature value for the first frequency.

42. The method according to claim 30, wherein the processing of the obtained in-phase and quadrature values by computed transmission tomography uses an iterative algorithm based on numerical optimization of a cost function.

43. The method according to claim 30, wherein the processing of the obtained in-phase and quadrature values by computed transmission tomography comprises processing the obtained in-phase and quadrature values by computed transmission tomography with model-based, iterative reconstruction techniques with or without phase-encoding.

44. An ultrasound computed tomography apparatus comprising:
an imaging area configured to accommodate a medium to be imaged;
an array of ultrasound elements arranged around the imaging area, the ultrasound elements comprising a transmitter positioned on a first side of the imaging area, and a plurality of receivers positioned on an opposite side of the imaging area;
respective receiver circuitry for each receiver of the plurality of receivers; and
a processing resource configured to perform computed transmission tomography; wherein:
the transmitter is configured to transmit through the imaging area a sequence of single-tone continuous-wave signals, the sequence of single-tone continuous-wave signals comprising at least one single-tone continuous-wave signal having a first frequency and at least one single-tone continuous-wave signal having a second, different frequency;
each of the plurality of receivers is configured to receive the sequence of single-tone continuous-wave signals that has been transmitted through the imaging area;
the respective receiver circuitry for each of the plurality of receivers is configured to mix each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and
the processing circuitry is configured to process the obtained in-phase and quadrature values by computed transmission tomography to obtain an image of the imaging area.

45. The apparatus according to claim 44, wherein the array of ultrasound elements comprises a plurality of transceivers each configured to transmit and receive ultrasound signals, the transmitter is a first transceiver of the plurality of transceivers and the receivers are further transceivers of the plurality of transceivers.

46. The apparatus according to claim 44, wherein the array is a circular or part-circular array.

47. The apparatus according to claim 44, wherein the array is moveable by a succession of positional increments relative to the medium and perpendicular to a plane of the transmitter and receivers.

48. The apparatus according to claim 44, wherein the array is a three-dimensional array comprising a plurality of two-dimensional arrays.

49. The apparatus according to claim 44, wherein the receiver circuitry comprises a mixer stage configured to mix each received single-tone continuous-wave signal of the sequence of single-tone continuous-wave signals with the respective single-frequency mixer signal having the same frequency as said single-tone continuous-wave signal to obtain the respective in-phase value and a respective quadrature value for said single-tone continuous-wave signal.

50. The apparatus according to claim 49, wherein the receiver circuitry further comprises a low-noise amplifier configured to amplify received signals, a pair of low-pass filters to remove frequencies above a threshold frequency, and a pair of analog to digital converters to digitize the in-phase and quadrature values.

51. The apparatus according to claim 50, wherein each analog to digital converter has a bit depth of at least 24 bits.

52. The apparatus according to claim 50, wherein a sampling rate of each analog to digital converter is between 1 kHz and 1000 kHz.

53. The apparatus according to claim 44, further comprising a controller configured to determine and/or store at least one of a respective voltage, a respective phase, a respective transmission time and a respective integration time for each of the single-tone continuous-wave signals, optionally wherein at least one of the respective voltages, respective phases, respective transmission times and respective integration times are programmable and frequency-dependent.

54. The apparatus according to claim 44, further comprising a signal generator configured to generate the sequence of single-tone continuous-wave signals.

55. An ultrasound computed tomography method comprising:
transmitting through a medium, by a transmitter, a sequence of single-tone continuous-wave signals, the sequence of single-tone continuous-wave signals comprising at least one single-tone continuous-wave signal having a first frequency and at least one single-tone continuous-wave signal having a second, different frequency;
receiving, by each of a plurality of receivers, the sequence of single-tone continuous-wave signals that has been transmitted through the medium;
for each of the plurality of receivers, mixing each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and
processing the obtained in-phase and quadrature values by computed transmission tomography to obtain an image of the medium.

56. The method according to claim 55, wherein at least one of:
the transmitter is positioned to a first side of the medium and the plurality of receivers is positioned to a second side of the medium, wherein the second side of the medium is different to the first side of the medium;
the second side of the medium is opposite, and optionally also lateral, to the first side of the medium;
the transmitter is positioned on a first surface in space and the plurality of receivers is positioned on a second surface in space; or
the transmitter is positioned on a first line in space and the plurality of receivers is positioned on a second line in space.

57. An ultrasound computed tomography apparatus comprising:
an imaging area configured to accommodate a medium to be imaged;
an array of ultrasound elements arranged around the imaging area, the ultrasound elements comprising a transmitter, and a plurality of receivers;
respective receiver circuitry for each receiver of the plurality of receivers; and
a processing resource configured to perform computed transmission tomography;
wherein
the transmitter is configured to transmit through the imaging area a sequence of single-tone continuous-wave signals, the sequence of single-tone continuous-wave signals comprising at least one single-tone continuous-wave signal having a first frequency and at least one single-tone continuous-wave signal having a second, different frequency;
each of the plurality of receivers is configured to receive the sequence of single-tone continuous-wave signals that has been transmitted through the imaging area;
the respective receiver circuitry for each of the plurality of receivers is configured to mix each received single-tone continuous-wave signal of the sequence of single-tone continuous-wave signals with a respective single-frequency mixer signal having a same frequency as said single-tone continuous-wave signal to obtain a respective in-phase value and a respective quadrature value for said single-tone continuous-wave signal; and
the processing circuitry is configured to process the obtained in-phase and quadrature values by computed transmission tomography to obtain an image of the imaging area.

58. The apparatus according to claim 57, wherein at least one of:
the transmitter is positioned to a first side of the imaging area and the plurality of receivers is positioned to a second side of the imaging area, wherein the second side of the imaging area is different to the first side of the imaging area;
the second side of the imaging area is opposite, and optionally also lateral, to the first side of the imaging area;
the transmitter is positioned on a first surface in space and the plurality of receivers is positioned on a second surface in space; or
the transmitter is positioned on a first line
